(19) United States
(12) Patent Application Publication
(10) Pub. No.: US 2026/0345741 A1
(43) Pub. Date: Nov. 5, 2026

(54) Pharmaceutical Composition Comprising Elastin and Method of Use Thereof

(71) Applicant: Gene Company (Pty)Ltd., Kelvin Grove (AU)

(72) Inventors: Kevin MORRIS, Big Bear City, CA (US); Yu-Hsin LIN, Sydney (AU)

(21) Appl. No.: 19/675,566

(22) Filed: May 13, 2026

Related U.S. Application Data
(63) Continuation of application No. PCT/US24/55795, filed on Nov. 13, 2024.
(60) Provisional application No. 63/598,499, filed on Nov. 13, 2023.
(60) Provisional application No. 63/607,552, filed on Dec. 7, 2023.
(60) Provisional application No. 63/688,874, filed on Aug. 30, 2024.

(51) Int. Cl. A61K 38/39; A61K 9/50; A61K 38/17; A61K 48/00

(52) U.S. Cl. CPC A61K 38/39; A61K 9/5068; A61K 38/1709; A61K 48/0033; A61K 48/005

(57) ABSTRACT

The present invention provides a pharmaceutical composition comprising an exosome encapsulating one or more cargo RNAs comprising a package RNA and one or more fusion proteins wherein the nucleotide sequence of the package RNA is at least about 80%, about 85%, about 90%, about 95% or about 100% identical to the nucleotide sequence of an elastin (ELN) gene or an elastin messenger RNA (mRNA) that also includes any isoforms. The present invention also provides a method of treatment of elastin deficient related illness in a subject using the pharmaceutical composition of the present invention.

### CROSS REFERENCE TO RELATED APPLICATIONS

The present application is a national stage filing under 35 U.S.C. § 371 of international PCT application, PCT/US24/55795, filed Nov. 13, 2024, which claims priority under 35 U.S.C. § 119 (e) to U.S. provisional patent application, U.S. Ser. No. 63/598,499, filed Nov. 13, 2023, U.S. provisional patent application, U.S. Ser. No. 63/607,552, filed Dec. 7, 2023 and U.S. provisional patent application, U.S. Ser. No. 63/688,874, filed Aug. 30, 2024, each of which is incorporated herein by reference.

### INCORPORATION BY REFERENCE OF A SEQUENCE LISTING XML

A Sequence Listing is provided herewith as a Sequence Listing XML, “Elastin_Delivered_by_Exosome_Platform.xml” created on Nov. 13, 2024 and having a size of 61,881 bytes. The contents of the Sequence Listing XML are incorporated by reference herein in their entirety.

### FIELD OF THE INVENTION

The present invention provides a polynucleotide composition capable of upregulating the expression of elastin (ELN) and method of use and treatment thereof.

### BACKGROUND OF THE INVENTION

The skin is an organ that functions as the outer layer of protection and is constantly under bombardment of insults from external factors, including burns, cuts, ultraviolet light (UV), infrared radiation 1, and overtime results in the loss of fidelity and tensile strength. Skin is made up predominantly of elastin and collagen which for the main fibers that form the extracellular matrix of skin. Elastin provides the elasticity in skin while collagen is responsible for tensile strength. Elastin is an extracellular matrix (ECM) protein responsible for the elastic recoil of many vertebrate tissues and is a long-lived protein that degrades slowly in humans with a ½ life of ~70 years¹. As such humans are born with all the elastin ¹ Shapiro S D, Endicott S K, Province M A, Pierce J A, Campbell E J. Marked longevity of human lung parenchymal elastic fibers deduced from prevalence of D-aspartate and nuclear weapons-related radiocarbon. J Clin Invest. 1991; 87 (5): 1828-34. doi: 10.1172/JCI115204. PubMed PMID: 2022748; PMCID: PMC295305. required for life and it is not readily replenished during the lifecycle of a typical human. Replacement of elastin on skin through topical or microneedle administration could be impacting in tissue and wound healing as well as correction of scarring, burned or saggy wrinkly skin. Therefore, there is a need for a therapeutically effective composition and method to upregulate expression of Elastin in humans without or mitigate negative effects on the skin.

### SUMMARY OF THE INVENTION

A pharmaceutical composition comprising an exosome encapsulating one or more cargo RNAs and one or more fusion proteins, wherein the fusion protein comprises an exosome-associated transmembrane protein fused to a packaging protein; wherein the cargo RNA comprises a package RNA and a packaging domain; wherein the packaging protein is capable of binding to the packaging domain to form a fusion protein cargo RNA complex; and wherein the package RNA comprises an elastin protein encoding polynucleotide wherein nucleotide sequence of the elastin protein encoding polynucleotide is at least about 80%, about 85%, about 90%, about 95% or about 100% identical to the nucleotide sequence of an elastin gene or an elastin messenger RNA (mRNA). The present invention also provides a method of treatment of elastin-associated disease of a subject comprising the step of administration of a therapeutically effective amount of the pharmaceutical composition of the present invention to the subject.

### BRIEF DESCRIPTION OF THE DRAWINGS

FIG. 1 illustrates an Elastin mRNA expressing plasmid. The Hygromycin selectable mRNA expressing plasmid expresses a recombinant ELN gene containing 3′ distal packaging domains that interact with L7ae and Ula are shown as Ula RBS and CD box, downstream of a stabilizing GC Morris motif.

FIG. 2 illustrates a CD63-L7ae RNA packaging plasmid p25. Blastocydin resistant CD63-L7ae expressing RNA packaging motif capable of binding and packaging CD domains in the 3′ tail of ELN (p16) The CD63 exosome specific fusion protein is expressed from this construct.

FIG. 3 illustrates a CD63-Ula RNA packaging plasmid p26. Blastocydin resistant CD63-U1a expressing RNA packaging motif capable of binding and packaging UR domains in the 3′ tail of ELN (p16). The CD63 exosome specific fusion protein is expressed from this construct.

FIG. 4 illustrates a PTGFRN-U1a RNA packaging plasmid p27. Puromycin resistant PTGFRN-U1a expressing RNA packaging motif capable of binding and packaging UR domains in the 3′ tail of ELN (p16). The PTGFRN exosome specific fusion protein is expressed from this construct.

FIG. 5 illustrates a CD81-Ula RNA packaging plasmid p28. Puromycin resistant CD81-Ula expressing RNA packaging motif capable of binding and packaging UR domains in the 3′ tail of ELN (p16). The PTGFRN exosome specific fusion protein is expressed from this construct.

FIG. 6 illustrates an EV enhancer plasmid p30. Neomycin resistant Connexin 43 and truncated Myoferlin expressing EV enhancer plasmid, based on works described in or provisional patent application entitled (Low Immunogenic Exosome-Based RNA Package and Delivery System, Composition and Method of Preparation Thereof).

FIG. 7 Illustrates various embodiments of the fusion protein encoding plasmids of the exosome-based RNA package and delivery system of the present invention for the delivery of GFP mRNA. CD63-L7ae+PTGFRN-Ula and CD81-Ula package and transfer GFP mRNA in EVs. Recipient cells were assessed for GFP mRNA compared to transient transfected producer HEK293 cells. The relative ratio of GFP mRNA in recipients was determined standardized to the producer cells at 48 hrs post culture in transwell assays with untreated HEK293 recipient cells. The average of triplicate treated cells is shown with the standard errors of the mean. Significant differences are demarcated by (**) with P<0.01 from a paired T-test relative to no packaging GFP control cells. Dual fusion protein packing GFP mRNA into exosomes. CD63-L7ae+PTGFRN-Ula and CD63-U1a+PTGFRN-Ula package GFP mRNAs into EVs, but efficiencies are within the range of the GFP control background non-specific packaging, e.g. no fusion protein present.

FIG. 8 Illustrates various embodiments of the exosome-based RNA package and delivery system of the present invention comprising two fusion protein encoding plasmids for the delivery of GFP mRNA.

### DETAILED DESCRIPTION OF THE INVENTION

As used in this specification and in claims which follow, the singular forms “a”, “an” and “the” include plural referents unless the context clearly indicates otherwise. Thus, for example, reference to “an ingredient” includes mixtures of ingredients, reference to “an active pharmaceutical agent” includes more than one active pharmaceutical agent, and the like.

As used herein, the term “about” as a modifier to a quantity is intended to mean+ or −20%, + or −15%, + or −10% or + or −5%, inclusive of the quantity being modified.

As used herein, the term “protein domain” or “domain” means a region of the polypeptide of a protein. In an embodiment, a protein domain is itself folded as a compact structure independent of rest polypeptide region. In an embodiment, the same or similar protein domain within different protein can share similar or identical functions. For example, ferlin proteins comprises multiple C2 domains and a transmembrane domain, and sometimes a Fer domain, DysF domain, or a combination thereof.

As used herein, the term “nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of nucleic acids contemplated herein include single and double stranded DNA, single and double stranded RNA, and hybrid molecules having mixtures of single and double stranded DNA and RNA. Examples of nucleic acids contemplated herein include any types of RNA (e.g., antisense RNA, mRNA, siRNA, miRNA, shRNA, guide RNA, dicer substrate RNA, dicer substrate siRNAs (dsiRNAs) (dsiRNA are cleaved by the RNase I class endoribonuclease dicer into 21-23 base duplexes having 2-base 3′-overhangs siRNA), and any type of DNA, genomic DNA, plasmid DNA, and minicircle DNA, and any fragments thereof. The term “duplex” in the context of nucleic acids refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. In an embodiment, the nucleotide sequence is provided using symbols ATCG (adenine (A), cytosine (C), guanine (G), and thymine (T)) for a DNA molecule, and provided with codes using symbols AUCG (adenine (A), cytosine (C), guanine (G), and uracil (U)) for a RNA molecule. In an embodiment, the symbols T and U are used interchangeably in a nucleotide sequence to illustrate the DNA and RNA molecule respectively made according to the nucleotide sequence.

As used herein, the terms “polypeptide,” “peptide” and “protein” generally refer to a polymer of amino acid residues. As used herein, the term also applies to amino acid polymers in which one or more amino acids are chemical analogs or modified derivatives of corresponding naturally occurring amino acids or are unnatural amino acids. The term “protein”, as generally used herein, refers to a polymer of amino acids linked to each other by peptide bonds to form a polypeptide for which the chain length is sufficient to produce tertiary and/or quaternary structure. In an embodiment, the “polypeptide,” “peptide” or “protein” of the present invention is prepared from a plasmid encoding said “polypeptide,” “peptide” or “protein”. Therefore, the “polypeptide,” “peptide” or “protein” of the present invention further comprises a nucleotide sequence encoding said “polypeptide,” “peptide” or “protein” that could be converted using a genetic code such as but not limited to the standard genetic code.

As used herein, “sequence identity” and “% identity,” refers to the value determined by comparing two optimally aligned sequences over a comparison window, wherein a portion of the sequence in the comparison window may comprise additions or deletions as compared to the reference sequence for optimal alignment of the two sequences. The number of positions at which identical amino acid residues occur in both sequences is determined, yielding the number of matched positions, which is divided by the total number of positions in the window of comparison and the result multiplied by 100 to yield the percentage of sequence identity. The comparison window is the entire length of the sequence being referred to unless indicated otherwise.

As used herein, “% similarity” is calculated as described for “% identity,” with the exception that the hydrophobic residues Ala, Val, Phe, Pro, Leu, Ile, Trp, Met, and Cys are similar; the basic residues Lys, Arg, and His are similar; the acidic residues Glu and Asp are similar; and the hydrophilic, uncharged residues Gln, Asn, Ser, Thr, and Tyr are similar. The remaining natural amino acid Gly is not similar to any other amino acid in this context.

As used herein, the term “subject,” “individual” or “patient” is used interchangeably herein, which refers to a vertebrate, preferably a mammal, more preferably a human. Mammals include, but are not limited to, murines, simians, humans, farm animals, sport animals, and pets.

As used herein, the term “effective amount” or “a therapeutically effective amount” of a drug, compound, pharmacologically active agent or a pharmaceutical composition comprises administering an amount thereof necessary to achieve a desired result. The exact amount required will vary from subject to subject, depending on the species, age, general condition of the subject, the severity of the disease, the particular active agent, its mode of administration, the desired outcome, and the like. In certain embodiments of the present invention, a “therapeutically effective amount” of a drug, compound, pharmacologically active agent or a pharmaceutical composition is that amount effective for increase expression of a protein by 10%, about 20%, about 30%, about 40%, about 50%, about 60%, about 70%, about 80%, about 90%, about 95%, about 100%, about 200%, 400%, 600%, 800%, 1000% or any percentage or percentage ranges falling within these values. In certain embodiments of the present invention, a “therapeutically effective amount” of a drug, compound, pharmacologically active agent or a pharmaceutical composition is that amount effective for increase expression of a protein so that the amount of that protein in a subject is restored to the same level in the subject as about 1 year, about 2 years, about 3 years, about 5 years, about 10 years, about 12 years, about 14 years, about 16 years, about 18 years, about 20 years, about 25 years, about 30 years, about 35 years, about 40 years about 50 years, about 60 years, about 70 years, about 80 years ago. In certain embodiments of the present invention, a “therapeutically effective amount” of a drug, compound, pharmacologically active agent or a pharmaceutical composition is that amount effective for inhibiting progression or reversing of any disease disclosed herein in a subject or a biological sample (e.g., in cells). In certain embodiments, disease progression is inhibited by about 10%, about 20%, about 30%, about 40%, about 50%, about 60%, about 70%, about 80%, about 90%, about 95%, or about 100% or any percentage or percentage ranges falling within these values. In certain embodiments, the drug, compound, pharmacologically active agent or a pharmaceutical composition inhibits disease progression by at least about 25%, at least about 50%, at least about 75%, at least about 90% or at least about 100% or any percentage or percentage ranges falling within these values. In certain embodiments of the present invention, a “therapeutically effective amount” refers to an amount of a drug, compound, pharmacologically active agent or a pharmaceutical composition sufficient to cause reversal of disease. In certain embodiments, the disease is reversed by about 1%, about 5%, about 10%, about 20%, about 30%, about 40%, about 50%, about 60%, about 70%, about 80%, about 90%, about 95%, about 100% or any percentage or percentage ranges falling within these values.

As used herein, the term “target” or “targeting” a polynucleotide comprises regulating the expression level, biological function, or a combination thereof, of said polynucleotide. In an embodiment, the regulation comprises direct regulation or indirect regulation. The indirect regulation comprises regulating the expression level, biological function, or a combination thereof, of the polynucleotide through an additional regulator. In an embodiment, the additional regulator comprises an antisense RNA.

As used herein, the term “ELN”, “elastin”, or “tropoelastin” is used interchangeably herein, which refers to the polynucleotides or polypeptides that encodes an elastin protein or any variants thereof.

The present invention provides one or more polynucleotides encoding an elastin protein or a variant thereof wherein the nucleotide sequence of each of the one or more polynucleotides is at least about 60%, about 65%, about 70%, about 75%, about 80%, about 85%, about 90%, about 95% or about 100% identical to the nucleotide sequence of an elastin gene (ELN) or an elastin messenger RNA (mRNA). In an embodiment, the elastin gene or mRNA can comprise any isoform thereof. In an embodiment, the elastin mRNA isoforms are derived from alternative splicing of the ELN gene. In an embodiment, the elastin protein encoding polynucleotide of the present invention encodes the elastin protein wherein the amino acid sequence of the elastin protein is at least about 80%, about 85%, about 90%, about 95% or about 100% identical or similar to SEQ ID NO. 1.

SEQ ID NO. 1:
MAGLTAAAPRPGVLLLLLSILHPSRPGGVPGAIPGGVPGGVFYPGAGLGA
LGGGALGPGGKPLKPVPGGLAGAGLGAGLGAFPAVTFPGALVPGGVADAA
AAYKAAKAGAGLGGVPGVGGLGVSAGAVVPQPGAGVKPGKVPGVGLPGVY
PGGVLPGARFPGVGVLPGVPTGAGVKPKAPGVGGAFAGIPGVGPFGGPQP
GVPLGYPIKAPKLPGGYGLPYTTGKLPYGYGPGGVAGAAGKAGYPTGTGV
GPQAAAAAAAKAAAKFGAGAAGVLPGVGGAGVPGVPGAIPGIGGIAGVGT
PAAAAAAAAAAKAAKYGAAAGLVPGGPGFGPGVVGVPGAGVPGVGVPGAG
IPVVPGAGIPGAAVPGVVSPEAAAKAAAKAAKYGARPGVGVGGIPTYGVG
AGGFPGFGVGVGGIPGVAGVPGVGGVPGVGGVPGVGISPEAQAAAAAKAA
KYGVGTPAAAAAKAAAKAAQFGLVPGVGVAPGVGVAPGVGVAPGVGLAPG
VGVAPGVGVAPGVGVAPGIGPGGVAAAAKSAAKVAAKAQLRAAAGLGAGI
PGLGVGVGVPGLGVGAGVPGLGVGAGVPGFGAVPGALAAAKAAKYGAAVP
GVLGGLGALGGVGIPGGVVGAGPAAAAAAAKAAAKAAQFGLVGAAGLGGL
GVGGLGVPGVGGLGGIPPAAAAKAAKYGAAGLGGVLGGAGQFPLGGVAAR
PGFGLSPIFPGGACLGKACGRKRK

In an embodiment, the elastin encoding polynucleotide of the present invention is about 500 to about 5000 nucleotides (nt) in length such as about 500 nt, about 750 nt, about 1000 nt, about 1250 nt, about 1500 nt, about 1750 nt, about 2000 nt, about 2250 nt, about 2500 nt, about 2750 nt, about 3000 nt, about 3250 nt, about 3500 nt, about 3750 nt, about 4000 nt, about 4250 nt, about 4500 nt, about 4750 nt, or about 5000 nt. In an embodiment, the elastin encoding polynucleotide of the present invention is about 500 to about 1000 nt in length, about 1000 to about 2000 nt in length, about 2000 to about 3000 nt in length, about 3000 to about 4000 nt in length, or about 4000 to about 5000 nt in length. In an embodiment, the nucleotide sequence of the elastin encoding polynucleotide of the present invention is at least about 60%, about 65%, about 70%, about 75%, about 80%, about 85%, about 90%, about 95% or about 100% identical to the nucleotide sequence of ENST00000252034.12, ENST00000380575.8, ENST00000429192.5, ENST00000357036.9, ENST00000380576.9, ENST00000445912.5, ENST00000320492.11, ENST00000414324.5, ENST00000380562.8, ENST00000380584.8, ENST00000458204.5, ENST00000621115.4, or ENST00000692049.1 deposited in the Ensembl genome browser². In an embodiment, the nucleotide sequence of the elastin encoding polynucleotide of the present invention is at least about 60%, about 65%, about 70%, about 75%, about 80%, about 85%, about 90%, about 95% or about 100% identical to the nucleotide sequence of CCDS 5562.2, CCDS 43598.1, CCDS 43599.1, CCDS 47611.1, CCDS 47612.1, CCDS 64673.1, CCDS 64674.1, CCDS 64675.1, CCDS 64676.1, CCDS 64677.1, CCDS 64678.1, CCDS 75616.1, or CCDS 75617.1 deposited in the NCBI CCDS Database³. In an embodiment, the nucleotide sequence of the elastin encoding polynucleotide of the present invention is at least about 60%, about 65%, about 70%, about 75%, about 80%, about 85%, about 90%, about 95% or about 100% identical to the nucleotide sequence of SEQ ID NO. 2. In an embodiment, the nucleotide sequence of the elastin encoding polynucleotide of the present invention is at least about 60%, about 65%, about 70%, about 75%, about 80%, about 85%, about 90%, about 95% or about 100% identical to the nucleotide sequence of SEQ ID NO. 3. ² http://asia.ensembl.org/index.html³ https://www.ncbi.nlm.nih.gov/CCDS/CcdsBrowse.cgi

SEQ ID NO. 2:
atggcgggcctgaccgcggcggcgccgcgcccgggcgtgctgctgctgctgctgagcattctgcatccgagccgcccgggcggc
gtgccgggcgcgattccgggcggcgtgccgggcggcgtgttttatccgggcgcgggcctgggcgcgctgggcggcggcgcgctg
ggcccgggcggcaaaccgctgaaaccggtgccgggcggcctggcgggcgcgggcctgggcgcgggcctgggcgcgtttccgg
cggtgacctttccgggcgcgctggtgccgggcggcgtggcggatgcggcggcggcgtataaagcggcgaaagcgggcgcgggc
ctgggcggcgtgccgggcgtgggcggcctgggcgtgagcgcgggcgcggtggtgccgcagccgggcgcgggcgtgaaaccgg
gcaaagtgccgggcgtgggcctgccgggcgtgtatccgggcggcgtgctgccgggcgcgcgctttccgggcgtgggcgtgctgc
cgggcgtgccgaccggcgcgggcgtgaaaccgaaagcgccgggcgtgggcggcgcgtttgcgggcattccgggcgtgggcccg
tttggcggcccgcagccgggcgtgccgctgggctatccgattaaagcgccgaaactgccgggcggctatggcctgccgtataccac
cggcaaactgccgtatggctatggcccgggcggcgtggcgggcgcggcgggcaaagcgggctatccgaccggcaccggcgtgg
gcccgcaggcggcggcggcggcggcggcgaaagcggcggcgaaatttggcgcgggcgcggcgggcgtgctgccgggcgtgg
gcggcgcgggcgtgccgggcgtgccgggcgcgattccgggcattggcggcattgcgggcgtgggcaccccggcggcggcggc
ggcggcggcggcggcggcgaaagcggcgaaatatggcgcggcggcgggcctggtgccgggcggcccgggctttggcccggg
cgtggtgggcgtgccgggcgcgggcgtgccgggcgtgggcgtgccgggcgcgggcattccggtggtgccgggcgcgggcattc
cgggcgcggcggtgccgggcgtggtgagcccggaagcggcggcgaaagcggcggcgaaagcggcgaaatatggcgcgcgcc
cgggcgtgggcgtgggcggcattccgacctatggcgtgggcgcgggcggctttccgggctttggcgtgggcgtgggcggcattcc
gggcgtggcgggcgtgccgggcgtgggcggcgtgccgggcgtgggcggcgtgccgggcgtgggcattagcccggaagcgcag
gcggcggcggcggcgaaagcggcgaaatatggcgtgggcaccccggcggcggcggcggcgaaagcggcggcgaaagcggc
gcagtttggcctggtgccgggcgtgggcgtggcgccgggcgtgggcgtggcgccgggcgtgggcgtggcgccgggcgtgggcc
tggcgccgggcgtgggcgtggcgccgggcgtgggcgtggcgccgggcgtgggcgtggcgccgggcattggcccgggcggcgt
ggcggcggcggcgaaaagcgcggcgaaagtggcggcgaaagcgcagctgcgcgcggcggcgggcctgggcgcgggcattcc
gggcctgggcgtgggcgtgggcgtgccgggcctgggcgtgggcgcgggcgtgccgggcctgggcgtgggcgcgggcgtgccg
ggctttggcgcggtgccgggcgcgctggcggcggcgaaagcggcgaaatatggcgcggcggtgccgggcgtgctgggcggcct
gggcgcgctgggcggcgtgggcattccgggcggcgtggtgggcgcgggcccggcggcggcggcggcggcggcgaaagcggc
ggcgaaagcggcgcagtttggcctggtgggcgcggcgggcctgggcggcctgggcgtgggcggcctgggcgtgccgggcgtgg
gcggcctgggcggcattccgccggcggcggcggcgaaagcggcgaaatatggcgcggcgggcctgggcggcgtgctgggcgg
cgcgggccagtttccgctgggcggcgtggcggcgcgcccgggctttggcctgagcccgatttttccgggcggcgcgtgcctgggca
aagcgtgcggccgcaaacgcaaagc
SEQ ID NO. 3 (ENST00000252034.12 (ELN) length = 3397, elastin coding sequence
shown in capital letters and 5′ and 3′ UTR in lowercase):
gcatttctccccgagATGGCGGGTCTGACGGCGGCGGCCCCGCGGCCCGGAGTCCTCCTG
CTCCTGCTGTCCATCCTCCACCCCTCTCGGCCTGGAGGGGTCCCTGGGGCCATTC
CTGGTGGAGTTCCTGGAGGAGTCTTTTATCCAGGGGCTGGTCTCGGAGCCCTTGG
AGGAGGAGCGCTGGGGCCTGGAGGCAAACCTCTTAAGCCAGTTCCCGGAGGGCT
TGCGGGTGCTGGCCTTGGGGCAGGGCTCGGCGCCTTCCCCGCAGTTACCTTTCCG
GGGGCTCTGGTGCCTGGTGGAGTGGCTGACGCTGCTGCAGCCTATAAAGCTGCTA
AGGCTGGCGCTGGGCTTGGTGGTGTCCCAGGAGTTGGTGGCTTAGGAGTGTCTGC
AGGTGCGGTGGTTCCTCAGCCTGGAGCCGGAGTGAAGCCTGGGAAAGTGCCGGG
TGTGGGGCTGCCAGGTGTATACCCAGGTGGCGTGCTCCCAGGAGCTCGGTTCCCC
GGTGTGGGGGTGCTCCCTGGAGTTCCCACTGGAGCAGGAGTTAAGCCCAAGGCT
CCAGGTGTAGGTGGAGCTTTTGCTGGAATCCCAGGAGTTGGACCCTTT
GGGGGACCGCAACCTGGAGTCCCACTGGGGTATCCCATCAAGGCCCCCAAGCTG
CCTGGTGGCTATGGACTGCCCTACACCACAGGGAAACTGCCCTATGGCTATGGGC
CCGGAGGAGTGGCTGGTGCAGCGGGCAAGGCTGGTTACCCAACAGGGACAGGG
GTTGGCCCCCAGGCAGCAGCAGCAGCGGCAGCTAAAGCAGCAGCAAAGTTCGGT
GCTGGAGCAGCCGGAGTCCTCCCTGGTGTTGGAGGGGCTGGTGTTCCTGGCGTGC
CTGGGGCAATTCCTGGAATTGGAGGCATCGCAGGCGTTGGGACTCCAGCTGCAG
CTGCAGCTGCAGCAGCAGCCGCTAAGGCAGCCAAGTATGGAGCTGCTGCAGGCT
TAGTGCCTGGTGGGCCAGGCTTTGGCCCGGGAGTAGTTGGTGTCCCAGGAGCTGG
CGTTCCAGGTGTTGGTGTCCCAGGAGCTGGGATTCCAGTTGTCCCAGGTGCTGGG
ATCCCAGGTGCTGCGGTTCCAGGGGTTGTGTCACCAGAAGCAGCTGCTAAG
GCAGCTGCAAAGGCAGCCAAATACGGGGCCAGGCCCGGAGTCGGAGTTGGAGG
CATTCCTACTTACGGGGTTGGAGCTGGGGGCTTTCCCGGCTTTGGTGTCGGAGTC
GGAGGTATCCCTGGAGTCGCAGGTGTCCCTGGTGTCGGAGGTGTTCCCGGAGTCG
GAGGTGTCCCGGGAGTTGGCATTTCCCCCGAAGCTCAGGCAGCAGCTGCCGCCA
AGGCTGCCAAGTACGGAGTGGGGACCCCAGCAGCTGCAGCTGCTAAAGCAGCCG
CCAAAGCCGCCCAGTTTGGGTTAGTTCCTGGTGTCGGCGTGGCTCCTGGAGTTGG
CGTGGCTCCTGGTGTCGGTGTGGCTCCTGGAGTTGGCTTGGCTCCTGGAGTTGGC
GTGGCTCCTGGAGTTGGTGTGGCTCCTGGCGTTGGCGTGGCTCCCGGCATTGGCC
CTGGTGGAGTTGCAGCTGCAGCAAAATCCGCTGCCAAGGTGGCTGCCAAAGCCC
AGCTCCGAGCTGCAGCTGGGCTTGGTGCTGGCATCCCTGGACTTGGAGTTGGTGT
CGGCGTCCCTGGACTTGGAGTTGGTGCTGGTGTTCCTGGACTTGGAGTTGGTGCT
GGTGTTCCTGGCTTCGGGGCAGTACCTGGAGCCCTGGCTGCCGCTAAAGCAGCCA
AATATGGAGCAGCAGTGCCTGGGGTCCTTGGAGGGCTCGGGGCTCTCGGTGGAG
TAGGCATCCCAGGCGGTGTGGTGGGAGCCGGACCCGCCGCCGCCGCTGCCGCAG
CCAAAGCTGCTGCCAAAGCCGCCCAGTTTGGCCTAGTGGGAGCCGCTGGGCTCG
GAGGACTCGGAGTCGGAGGGCTTGGAGTTCCAGGTGTTGGGGGCCTTGGAGGTA
TACCTCCAGCTGCAGCCGCTAAAGCAGCTAAATACGGTGCTGCTGGCCTTGGAGG
TGTCCTAGGGGGTGCCGGGCAGTTCCCACTTGGAGGAGTGGCAGCAAGACCTGG
CTTCGGATTGTCTCCCATTTTCCCAGGTGGGGCCTGCCTGGGGAAAGCTTGTGGC
CGGAAGAGAAAATGAgcttcctaggacccctgactcacgacctcatcaacgttggtgctactgcttggtggagaatgtaa
accctttgtaaccccatcccatgcccctccgactccccaccccaggagggaacgggcaggccgggcggccttgcagatccacagg
gcaaggaaacaagaggggagcggccaagtgccccgaccaggaggccccctacttcagaggcaagggccatgtggtcctggcccc
ccaccccatcccttcccacctaggagctccccctcacacagcctccatctccaggggaacttggtgctacacgctggtgctcttatcttc
ctggggggagggaggagggaagggtggcccctcggggaaccccctacctggggctcctctaaagatggtgcagacacttcctggg
cagtcccagctccccctgcccaccaggacccaccgttggctgccatccagttggtacccaagcacctgaagcctcaaagctggattc
gctctagcatccctectctcctgggtccacttggccgtctcctccccaccgatcgctgttccccacatctggggcgcttttgggttggaaa
accaccccacactgggaatagccaccttgcccttgtagaatccatccgcccatccgtccattcatccatcggtccgtccatccatgtccc
cagttgaccgcccggcaccactagctggctgggtgcacccaccatcaacctggttgacctgtcatggccgcctgtgccctgcctccac
ccccatcctacactcccccagggcgtgcggggctgtgcagactgggtgccaggcatctcctccccacccggggtgtccccacatgc
agtactgtataccccccatccctccctcggtccactgaacttcagagcagttcccattcctgccccgcccatctttttgtgtctcgctgtgat
agatcaataaatattttattttttgtcctggatatttggggattatttttgattgttgatattctcttttggttttattgttgtggttcattgaaaaaaaa
agataatttttttttctgatccggggagctgtatccccagtagaaaaaacattttaatcactctaatataactctggatgaaacacaccttttttt
ttaataagaaaagagaattaactgcttcagaaatgactaataaatgaaaaacctttaaaggaaa

The present invention also provides a pharmaceutical composition comprising a nanoparticle encapsulating any embodiment of the one or more of the elastin encoding polynucleotides of the present invention. In an embodiment, the nanoparticle comprises chemical nanoparticles such as but not limited to lipid nanoparticle, polymer nanoparticle, lipid-polymer hybrid nanoparticle and biological nanoparticles such as but not limited to liposome, exosome, virus or virus-like particle.

In an embodiment, the nanoparticle comprises an exosome. In an embodiment, the exosome comprises one or more fusion protein and one or more cargo RNA. In an embodiment, the one or more fusion protein each comprises an exosome associated transmembrane protein fused to a packaging protein. In an embodiment, the package protein is endogenous to a subject. In an embodiment, the one or more cargo RNA each comprises a package RNA and a packaging domain. In an embodiment, the package RNA comprises any embodiment of the elastin protein encoding polynucleotide of the present invention. In an embodiment, the nanoparticle comprises an exosome prepared from an exosome-based packaging and delivery system. In an embodiment, the system comprises a low immunogenic exosome-based packaging and delivery system. In an embodiment, the exosome-based RNA package and delivery system comprises an exosome producing cell, a cargo RNA encoding plasmid and one or more fusion protein encoding plasmids. In an embodiment, the cargo RNA comprises a package RNA and a packaging domain wherein the package RNA comprises any embodiment of the elastin protein encoding polynucleotides of the present invention. In an embodiment, the fusion protein comprises an exosome associated transmembrane protein fused to a packaging protein. In an embodiment, the exosome producing cell is capable of expressing the cargo RNA when it is transfected with the cargo RNA encoding plasmid. In an embodiment, the exosome producing cell is capable of expressing the fusion protein when it is transfected with the fusion protein encoding plasmid. In an embodiment, the exosome associated transmembrane protein comprises CD9, CD37, CD53, CD63, CD68, CD81, CD82, LAMP-1, LAMP-2A, LAMP-2B, LAMP-2C, lactadherin, or PTGFRN. In an embodiment, the packaging protein comprises an RNA-binding protein. In an embodiment, the packaging protein is endogenous to a subject. Various embodiments for the recombinant fusion protein comprising an exosome associated transmembrane protein and a packaging protein were disclosed in PCT application no. PCT/US2021/026892 filed 12 Apr. 2021 as well as US provisional patent application U.S. 63/598,499, the contents of which are incorporated by reference herein in their entireties. In an embodiment, the packaging protein comprises Ula protein.

In an embodiment the one or more fusion proteins comprise CD63-U1a protein. In an embodiment, nucleotide sequence of the CD63-U1a fusion protein of the present invention is at least about 80%, about 85%, about 90%, about 95% or about 100% identical to SEQ ID NO. 4. In an embodiment, the amino the acid sequence of the CD63-U1a fusion protein is at least about 80%, about 85%, about 90%, about 95% or about 100% identical or similar to SEQ ID NO. 5. In an embodiment the one or more fusion proteins comprise CD81-U1a protein. In an embodiment, nucleotide sequence of the CD81-U1a fusion protein of the present invention is at least about 80%, about 85%, about 90%, about 95% or about 100% identical to SEQ ID NO. 6. In an embodiment, the amino the acid sequence of the CD81-U1a fusion protein is at least about 80%, about 85%, about 90%, about 95% or about 100% identical or similar to SEQ ID NO. 7. In an embodiment the one or more fusion proteins comprise PTGFRN-U1a protein. In an embodiment, nucleotide sequence of the PTGFRN-U1a fusion protein of the present invention is at least about 80%, about 85%, about 90%, about 95% or about 100% identical or similar to SEQ ID NO. 8. In an embodiment, the amino the acid sequence of the PTGFRN-U1a fusion protein is about 80%, about 85%, about 90%, about 95% or about 100% identical to SEQ ID NO. 9.

SEQ ID NO. 4:
ATGGCGGTGGAAGGAGGAATGAAATGTGTGAAGTTCTTGCTCTACGTCCTCCTGC
TGGCCTTTTGCGCCTGTGCAGTGGGACTGATTGCCGTGGGTGTCGGGGCACAGCT
TGTCCTGAGTCAGACCATAATCCAGGGGGCTACCCCTGGCTCTCTGTTGCCAGTG
GTCATCATCGCAGTGGGTGTCTTCCTCTTCCTGGTGGCTTTTGTGGGCTGCTGCGG
GGCCTGCAAGGAGAACTATTGTCTTATGATCACGTTTGCCATCTTTCTGTCTCTTA
TCATGTTGGTGGAGGTGGCCGCAGCCATTGCTGGCTATGTGTTTAGAGATAAGGT
GATGTCAGAGTTTAATAACAACTTCCGGCAGCAGATGGAGAATTACCCGAAAAA
CAACCACACTGCTTCGATCCTGGACAGGATGCAGGCAGATTTTAAGTGCTGTGGG
GCTGCTAACTACACAGATTGGGAGAAAATCCCTTCCATGTCGAAGAACCGAGTC
CCCGACTCCTGCTGCATTAATGTTACTGTGGGCTGTGGGATTAATTTCAACGAGA
AGGCGATCCATAAGGAGGGCTGTGTGGAGAAGATTGGGGGCTGGCTGAGGAAA
AATGTGCTGGTGGTAGCTGCAGCAGCCCTTGGAATTGCTTTTGTCGAGGTTTTGG
GAATTGTCTTTGCCTGCTGCCTCGTGAAGAGTATCAGAAGTGGCTACGAGGTGAT
GgaattcggcggaggcgggtccATGGCAGTTCCCGAGACCCGCCCTAACCACACTATTTA
TATCAACAACCTCAATGAGAAGATCAAGAAGGATGAGCTAAAAAAGTCCCT
GTACGCCATCTTCTCCCAGTTTGGCCAGATCCTGGATATCCTGGTATCACGG
AGCCTGAAGATGAGGGGCCAGGCCTTTGTCATCTTCAAGGAGGTCAGCAGC
GCCACCAACGCCCTGCGCTCCATGCAGGGTTTCCCTTTCTATGACAAACCTA
TGCGTATCCAGTATGCCAAGACCGACTCAGATATCATTGCCAAGATGAAA
Uppercase sequence denotes CD63, lower case is linker sequence, and bold sequence
is the U1 small nuclear ribonucleoprotein polypeptide A (U1 snRNP A).
SEQ ID NO. 5:
MAVEGGMKCVKFLLYVLLLAFCACAVGLIAVGVGAQLVLSQTIIQGATPGSLLPVVI
IAVGVFLFLVAFVGCCGACKENYCLMITFAIFLSLIMLVEVAAAIAGYVFRDKVMSEF
NNNFRQQMENYPKNNHTASILDRMQADFKCCGAANYTDWEKIPSMSKNRVPDSCCI
NVTVGCGINFNEKAIHKEGCVEKIGGWLRKNVLVVAAAALGIAFVEVLGIVFACCLV
KSIRSGYEVMefggggsMAVPETRPNHTIYINNLNEKIKKDELKKSLYAIFSQFGQIL
DILVSRSLKMRGQAFVIFKEVSSATNALRSMQGFPFYDKPMRIQYAKTDSDIIAK
MK
Uppercase sequence denotes CD63, lower case is linker sequence, and bold sequence
is the U1 small nuclear ribonucleoprotein polypeptide A (U1 snRNP A).
SEQ ID NO. 6:
ATGTCCGGACTCAGATCTCGAGCTCAAGCTTCCGGAGTGGAGGGCTGCACCAAG
TGCATCAAGTACCTGCTCTTCGTCTTCAATTTCGTCTTCTGGCTGGCTGGAGGCGT
GATCCTGGGTGTGGCCCTGTGGCTCCGCCATGACCCGCAGACCACCAACCTCCTG
TATCTGGAGCTGGGAGACAAGCCCGCGCCCAACACCTTCTATGTAGGCATCTACA
TCCTCATCGCTGTGGGCGCTGTCATGATGTTCGTTGGCTTCCTGGGCTGCTACGGG
GCCATCCAGGAATCCCAGTGCCTGCTGGGGACGTTCTTCACCTGCCTGGTCATCC
TGTTTGCCTGTGAGGTGGCCGCCGGCATCTGGGGCTTTGTCAACAAGGACCAGAT
CGCCAAGGATGTGAAGCAGTTCTATGACCAGGCCCTACAGCAGGCCGTGGTGGA
TGATGACGCCAACAACGCCAAGGCTGTGGTGAAGACCTTCCACGAGACGCTTGA
CTGCTGTGGCTCCAGCACACTGACTGCTTTGACCACCTCAGTGCTCAAGAACAAT
TTGTGTCCCTCGGGCAGCAACATCATCAGCAACCTCTTCAAGGAGGACTGCCACC
AGAAGATCGATGACCTCTTCTCCGGGAAGCTGTACCTCATCGGCATTGCTGCCAT
CGTGGTCGCTGTGATCATGATCTTCGAGATGATCCTGAGCATGGTGCTGTGCTGT
GGCATCCGGAACAGCTCCGTGTACGAATTCGGCGGAGGCGGGTCCATGGCAGTT
CCCGAGACCCGCCCTAACCACACTATTTATATCAACAACCTCAATGAGAAGATCA
AGAAGGATGAGCTAAAAAAGTCCCTGTACGCCATCTTCTCCCAGTTTGGCCAGAT
CCTGGATATCCTGGTATCACGGAGCCTGAAGATGAGGGGCCAGGCCTTTGTCATC
TTCAAGGAGGTCAGCAGCGCCACCAACGCCCTGCGCTCCATGCAGGGTTTCCCTT
TCTATGACAAACCTATGCGTATCCAGTATGCCAAGACCGACTCAGATATCATTGC
CAAGATGAAATAA
SEQ ID NO. 7:
MSGLRSRAQASGVEGCTKCIKYLLFVFNFVFWLAGGVILGVALWLRHDPQTTNLLY
LELGDKPAPNTFYVGIYILIAVGAVMMFVGFLGCYGAIQESQCLLGTFFTCLVILFAC
EVAAGIWGFVNKDQIAKDVKQFYDQALQQAVVDDDANNAKAVVKTFHETLDCCG
SSTLTALTTSVLKNNLCPSGSNIISNLFKEDCHQKIDDLFSGKLYLIGIAAIVVAVIMIFE
MILSMVLCCGIRNSSVYEFGGGGSMAVPETRPNHTIYINNLNEKIKKDELKKSLYAIF
SQFGQILDILVSRSLKMRGQAFVIFKEVSSATNALRSMQGFPFYDKPMRIQYAKTDSD
IIAKMK
SEQ ID NO. 8:
ATGGGGCGCCTGGCCTCCAGGCCGCTGCTGCTGGCGCTCCTGTCGTTGGCTCTTT
GCCGAGGGCGTGTGGTGAGAGTCCCCACAGCGACCCTGGTTCGAGTGGTGGGCA
CTGAGCTGGTCATCCCCTGCAACGTCAGTGACTATGATGGCCCCAGCGAGCAAA
ACTTTGACTGGAGCTTCTCATCTTTGGGGAGCAGCTTTGTGGAGCTTGCAAGCAC
CTGGGAGGTGGGGTTCCCAGCCCAGCTGTACCAGGAGCGGCTGCAGAGGGGCGA
GATCCTGTTAAGGCGGACTGCCAACGACGCCGTGGAGCTCCACATAAAGAACGT
CCAGCCTTCAGACCAAGGCCACTACAAATGTTCAACCCCCAGCACAGATGCCAC
TGTCCAGGGAAACTATGAGGACACAGTGCAGGTTAAAGTGCTGGCCGACTCCCT
GCACGTGGGCCCCAGCGCGCGGCCCCCGCCGAGCCTGAGCCTGCGGGAGGGGGA
GCCCTTCGAGCTGCGCTGCACCGCCGCCTCCGCCTCGCCGCTGCACACGCACCTG
GCGCTGCTGTGGGAGGTGCACCGCGGCCCGGCCAGGCGGAGCGTCCTCGCCCTG
ACCCACGAGGGCAGGTTCCACCCGGGCCTGGGGTACGAGCAGCGCTACCACAGT
GGGGACGTGCGCCTCGACACCGTGGGCAGCGACGCCTACCGCCTCTCAGTGTCC
CGGGCTCTGTCTGCCGACCAGGGCTCCTACAGGTGTATCGTCAGCGAGTGGATCG
CCGAGCAGGGCAACTGGCAGGAAATCCAAGAAAAGGCCGTGGAAGTTGCCACC
GTGGTGATCCAGCCATCAGTTCTGCGAGCAGCTGTGCCCAAGAATGTGTCTGTGG
CTGAAGGAAAGGAACTGGACCTGACCTGTAACATCACAACAGACCGAGCCGATG
ACGTCCGGCCCGAGGTGACGTGGTCCTTCAGCAGGATGCCTGACAGCACCCTAC
CTGGCTCCCGCGTGTTGGCGCGGCTTGACCGTGATTCCCTGGTGCACAGCTCGCC
TCATGTTGCTTTGAGTCATGTGGATGCACGCTCCTACCATTTACTGGTTCGGGATG
TTAGCAAAGAAAACTCTGGCTACTATTACTGCCACGTGTCCCTGTGGGCACCCGG
ACACAACAGGAGCTGGCACAAAGTGGCAGAGGCCGTGTCTTCCCCAGCTGGTGT
GGGTGTGACCTGGCTAGAACCAGACTACCAGGTGTACCTGAATGCTTCCAAGGTC
CCCGGGTTTGCGGATGACCCCACAGAGCTGGCATGCCGGGTGGTGGACACGAAG
AGTGGGGAGGCGAATGTCCGATTCACGGTTTCGTGGTACTACAGGATGAACCGG
CGCAGCGACAATGTGGTGACCAGCGAGCTGCTTGCAGTCATGGACGGGGACTGG
ACGCTAAAATATGGAGAGAGGAGCAAGCAGCGGGCCCAGGATGGAGACTTTATT
TTTTCTAAGGAACATACAGACACGTTCAATTTCCGGATCCAAAGGACTACAGAGG
AAGACAGAGGCAATTATTACTGTGTTGTGTCTGCCTGGACCAAACAGCGGAACA
ACAGCTGGGTGAAAAGCAAGGATGTCTTCTCCAAGCCTGTTAACATATTTTGGGC
ATTAGAAGATTCCGTGCTTGTGGTGAAGGCGAGGCAGCCAAAGCCTTTCTTTGCT
GCCGGAAATACATTTGAGATGACTTGCAAAGTATCTTCCAAGAATATTAAGTCGC
CACGCTACTCTGTTCTCATCATGGCTGAGAAGCCTGTCGGCGACCTCTCCAGTCC
CAATGAAACGAAGTACATCATCTCTCTGGACCAGGATTCTGTGGTGAAGCTGGA
GAATTGGACAGATGCATCACGGGTGGATGGCGTTGTTTTAGAAAAAGTGCAGGA
GGATGAGTTCCGCTATCGAATGTACCAGACTCAGGTCTCAGACGCAGGGCTGTAC
CGCTGCATGGTGACAGCCTGGTCTCCTGTCAGGGGCAGCCTTTGGCGAGAAGCA
GCAACCAGTCTCTCCAATCCTATTGAGATAGACTTCCAAACCTCAGGTCCTATAT
TTAATGCTTCTGTGCATTCAGACACACCATCAGTAATTCGGGGAGATCTGATCAA
ATTGTTCTGTATCATCACTGTCGAGGGAGCAGCACTGGATCCAGATGACATGGCC
TTTGATGTGTCCTGGTTTGCGGTGCACTCTTTTGGCCTGGACAAGGCTCCTGTGCT
CCTGTCTTCCCTGGATCGGAAGGGCATCGTGACCACCTCCCGGAGGGACTGGAA
GAGCGACCTCAGCCTGGAGCGCGTGAGTGTGCTGGAATTCTTGCTGCAAGTGCAT
GGCTCCGAGGACCAGGACTTTGGCAACTACTACTGTTCCGTGACTCCATGGGTGA
AGTCACCAACAGGTTCCTGGCAGAAGGAGGCAGAGATCCACTCCAAGCCCGTTT
TTATAACTGTGAAGATGGATGTGCTGAACGCCTTCAAGTATCCCTTGCTGATCGG
CGTCGGTCTGTCCACGGTCATCGGGCTCCTGTCCTGTCTCATCGGGTACTGCAGCT
CCCACTGGTGTTGTAAGAAGGAGGTTCAGGAGACACGGCGCGAGCGCCGCAGGC
TCATGTCGATGGAGATGGACGAATTCGGCGGAGGCGGGTCCATGGCAGTTCCCG
AGACCCGCCCTAACCACACTATTTATATCAACAACCTCAATGAGAAGATCAAGA
AGGATGAGCTAAAAAAGTCCCTGTACGCCATCTTCTCCCAGTTTGGCCAGATCCT
GGATATCCTGGTATCACGGAGCCTGAAGATGAGGGGCCAGGCCTTTGTCATCTTC
AAGGAGGTCAGCAGCGCCACCAACGCCCTGCGCTCCATGCAGGGTTTCCCTTTCT
ATGACAAACCTATGCGTATCCAGTATGCCAAGACCGACTCAGATATCATTGCCAA
GATGAAATAG
SEQ ID NO. 9:
MGRLASRPLLLALLSLALCRGRVVRVPTATLVRVVGTELVIPCNVSDYDGPSEQNFD
WSFSSLGSSFVELASTWEVGFPAQLYQERLQRGEILLRRTANDAVELHIKNVQPSDQ
GHYKCSTPSTDATVQGNYEDTVQVKVLADSLHVGPSARPPPSLSLREGEPFELRCTA
ASASPLHTHLALLWEVHRGPARRSVLALTHEGRFHPGLGYEQRYHSGDVRLDTVGS
DAYRLSVSRALSADQGSYRCIVSEWIAEQGNWQEIQEKAVEVATVVIQPSVLRAAVP
KNVSVAEGKELDLTCNITTDRADDVRPEVTWSFSRMPDSTLPGSRVLARLDRDSLVH
SSPHVALSHVDARSYHLLVRDVSKENSGYYYCHVSLWAPGHNRSWHKVAEAVSSP
AGVGVTWLEPDYQVYLNASKVPGFADDPTELACRVVDTKSGEANVRFTVSWYYR
MNRRSDNVVTSELLAVMDGDWTLKYGERSKQRAQDGDFIFSKEHTDTFNFRIQRTT
EEDRGNYYCVVSAWTKQRNNSWVKSKDVFSKPVNIFWALEDSVLVVKARQPKPFF
AAGNTFEMTCKVSSKNIKSPRYSVLIMAEKPVGDLSSPNETKYIISLDQDSVVKLENW
TDASRVDGVVLEKVQEDEFRYRMYQTQVSDAGLYRCMVTAWSPVRGSLWREAAT
SLSNPIEIDFQTSGPIFNASVHSDTPSVIRGDLIKLFCIITVEGAALDPDDMAFDVSWFA
VHSFGLDKAPVLLSSLDRKGIVTTSRRDWKSDLSLERVSVLEFLLQVHGSEDQDFGN
YYCSVTPWVKSPTGSWQKEAEIHSKPVFITVKMDVLNAFKYPLLIGVGLSTVIGLLSC
LIGYCSSHWCCKKEVQETRRERRRLMSMEMDEFGGGGSMAVPETRPNHTIYINNLN
EKIKKDELKKSLYAIFSQFGQILDILVSRSLKMRGQAFVIFKEVSSATNALRSMQGFPF
YDKPMRIQYAKTDSDIIAKMK

In an embodiment, the cargo RNA comprises a package RNA and a packaging domain capable of binding to the packaging protein of the fusion protein, wherein the package RNA comprises any embodiment of the one or more elastin encoding polynucleotides of the present invention. In an embodiment, the packaging domain comprises UR domain. In an embodiment the nucleotide sequence of the UR domain is at least about 80%, about 85%, about 90%, about 95% or about 100% identical to SEQ ID NO. 10 or SEQ ID NO. 11. In an embodiment, the cargo RNA further comprises stabilizing domains. In an embodiment, the stabilizing domain comprises OH domain and/or MorrisMotif domain wherein the nucleotide sequence of the OH domain is at least about 80%, about 85%, about 90%, about 95% or about 100% identical to SEQ ID NO. 12 and the nucleotide sequence of the MorrisMotif domain is at least about 80%, about 85%, about 90%, about 95% or about 100% identical to SEQ ID NO. 13.

SEQ ID NO. 10: AATCCATTGCACTCCGGATT
SEQ ID NO. 11: AATCCATTGCACTCCGGATTT
SEQ ID NO. 12: CTGCAGATATCCAGCACAGTGGC
SEQ ID NO. 13: GCGCAGCGCGCGCAGCGC

In an embodiment, the cargo RNA further comprises a SINE-derived nuclear RNA LOcalizatIoN (SIRLOIN) sequence wherein the SIRLOIN sequence is upstream of the UR sequence of the cargo RNA. In an embodiment, the nucleotide sequence of SIRLOIN sequence is at least about 80%, about 85%, about 90%, about 95% or about 100% identical to SEQ ID NO. 14.

SEQ ID NO. 14:
CGCCTCCCGGGTTCAAGCGATTCTCCTGCCTCAGCCTCCCGA

In an embodiment, the cargo RNA comprising an elastin mRNA, one or more UR domains and one or more stabilizing domains comprises a nucleotide sequence at least about 80%, about 85%, about 90%, about 95% or about 100% identical to SEQ ID NO. 15.

SEQ ID NO. 15:
atggcgggcctgaccgcggcggcgccgcgcccgggcgtgctgctgctgct
gctgagcattctgcatccgagccgcccgggcggcgtgccgggcgcgattc
cgggcggcgtgccgggcggcgtgttttatccgggcgcgggcctgggcgcg
ctgggcggcggcgcgctgggcccgggcggcaaaccgctgaaaccggtgcc
gggcggcctggcgggcgcgggcctgggcgcgggcctgggcgcgtttccgg
cggtgacctttccgggcgcgctggtgccgggcggcgtggcggatgcggcg
gcggcgtataaagcggcgaaagcgggcgcgggcctgggcggcgtgccggg
cgtgggcggcctgggcgtgagcgcgggcgcggtggtgccgcagccgggcg
cgggcgtgaaaccgggcaaagtgccgggcgtgggcctgccgggcgtgtat
ccgggcggcgtgctgccgggcgcgcgctttccgggcgtgggcgtgctgcc
gggcgtgccgaccggcgcgggcgtgaaaccgaaagcgccgggcgtgggcg
gcgcgtttgcgggcattccgggcgtgggcccgtttggcggcccgcagccg
ggcgtgccgctgggctatccgattaaagcgccgaaactgccgggcggcta
tggcctgccgtataccaccggcaaactgccgtatggctatggcccgggcg
gcgtggcgggcgcggcgggcaaagcgggctatccgaccggcaccggcgtg
ggcccgcaggcggcggcggcggcggcggcgaaagcggcggcgaaatttgg
cgcgggcgcgggggcgtgctgccgggcgtgggcggcgcgggcgtgccggg
cgtgccgggcgcgattccgggcattggcggcattgcgggcgtgggcaccc
cggcggcggcggcggcggcggcggcggcggcgaaagcggcgaaatatggc
gcggcggcgggcctggtgccgggcggcccgggctttggcccgggcgtggt
gggcgtgccgggcgcgggcgtgccgggcgtgggcgtgccgggcgcgggca
ttccggtggtgccgggcgcgggcattccgggcgcggcggtgccgggcgtg
gtgagcccggaagcggcggcgaaagcggcggcgaaagcggcgaaatatgg
cgcgcgcccgggcgtgggcgtgggcggcattccgacctatggcgtgggcg
cgggcggctttccgggctttggcgtgggcgtgggcggcattccgggcgtg
gcgggcgtgccgggcgtgggcggcgtgccgggcgtgggggcgtgccgggc
gtgggcattagcccggaagcgcaggcggcggcggcggcgaaagcggcgaa
atatggcgtgggcaccccggggggggcggcgaaagcggcggcgaaagcgg
cgcagtttggcctggtgccgggcgtgggcgtggcgccgggcgtgggcgtg
gcgccgggcgtgggcgtggcgccgggcgtgggcctggcgccgggcgtggg
cgtggcgccgggcgtgggcgtggcgccgggcgtgggcgtggcgccgggca
ttggcccgggcggcgtggcggcggcggcgaaaagcgcggcgaaagtggcg
gcgaaagcgcagctgcgcgcggcggcgggcctgggcgcgggcattccggg
cctgggcgtgggcgtgggcgtgccgggcctgggcgtgggcgcgggcgtgc
cgggcctgggcgtgggcgcgggcgtgccgggctttggcgcggtgccgggc
gcgctggcggcggcgaaagcggcgaaatatggcgcggcggtgccgggcgt
gctgggcggcctgggcgcgctgggcggcgtgggcattccgggcggcgtgg
tgggcgcgggcccggcggcggcggcggcggcggcgaaagcggcggcgaaa
gcggcgcagtttggcctggtgggcgcggcgggcctgggcggcctgggcgt
gggcggcctgggcgtgccgggcgtgggcggcctgggcggcattccgccgg
cggcggcggcgaaagcggcgaaatatggcgcggcgggcctgggcggcgtg
ctgggcggcgcgggccagtttccgctgggcggcgtggcggcgcgcccggg
ctttggcctgagcccgatttttccgggcggcgcgtgcctgggcaaagcgt
gcggccgcaaacgcaaaGCGCAGCGCGCGCAGCGCatagactttgggtac
cgtgatccgaaaggtgagtacccGTGAGATCCgggtaccgtgatccgaaa
ggtgagtacccTTCGAATCCATTGCACTCCGGATTTTTGAATTGGAATCC
ATTGCACTCCGGATTATCCTTCGCTGCAGATATCCAGCACAGTGGCatag
GCGCAGCGCGCGCAGCGCactttgggtaccgtgatccgaaaggtgagtac
ccGGATCCgggtaccgtgatccgaaaggtgagtacccTTCGAATCCATTG
CACTCCGGATTTTTGAATTGGAATCCATTGCACTCCGGATTATCCTTCGC
TGCAGATATCCAGCACAGTGGC

In an embodiment, the pharmaceutical composition comprising a nanoparticle encapsulating any embodiment of the one or more elastin protein encoding polynucleotides of the present invention further comprises a nanoparticle payload release enhancer. In an embodiment, the nanoparticle release enhancer enhances the release of payload from the nanoparticle by at least about 2-fold, at least about 5-fold, at least about 10-fold, at least about 15-fold, at least about 20-fold, or at least about 25-fold.

In an embodiment, the nanoparticle payload release enhancer comprises a modified myoferlin protein. In an embodiment, the modified myoferlin protein comprises C2F, C2G, transmembrane domain, or a combination thereof. In an embodiment, the modified myoferlin protein consists of C2F, C2G, transmembrane domain. In an embodiment, the modified myoferlin protein consists of domains selected from the group consisting of C2F, C2G, and transmembrane domains. In an embodiment, the amino acid sequence of the C2F domain is at least about 80%, about 85%, about 90%, about 95% or about 100% identical or similar to SEQ ID NO. 16. In an embodiment, the amino acid sequence of the C2G domain is at least about 80%, about 85%, about 90%, about 95% or about 100% identical or similar to SEQ ID NO. 17. In an embodiment, the amino acid sequence of the transmembrane domain is at least about 80%, about 85%, about 90%, about 95% or about 100% identical or similar to SEQ ID NO. 18.

SEQ ID NO. 16 (C2F):
QFRELPDSVPQECTVRIYIVRGLELQPQDNNGLCDPYIKITLGKKVIEDR
DHYIPNTLNPVFGRMYELSCYLPQEKDLKISVYDYDTFTRDEKVGETIID
LENRFLSRFGSHCGIPEEYCVSGV
SEQ ID NO. 17 (C2G):
PFNITPRKAKKYYLRVIIWNTKDVILDEKSITGEEMSDIYVKGWVPGNEE
NKQKTDVHYRSLDGEGNFNWRFVFPFDYLPAEQLCIVAKKEHFWSIDQTE
FRIPPRLIIQIWDNDKFSLDDYLGFLELDLRH
SEQ ID NO. 18 (transmembrane domain):
PDLKAMNPLKAKTASLFEQKSMKGWWPCYAEKDGARVMAGKVEMTLEILN
EKEADERPAGKGRDEPNMNPKLDLPNRPETSFLWFTNPCKTMKFIVWRRF
KWVIIGLLFLLILLLFVAV

In an embodiment, the modified myoferlin protein comprises C2A, FerA, FerB, DysFN, transmembrane domain, or a combination thereof. In an embodiment, the modified myoferlin protein consists of C2A, FerA, FerB, DysFN, transmembrane domain. In an embodiment, the modified myoferlin protein consists of domains selected from the group consisting of C2A, FerA, FerB, DysFN and transmembrane domain. In an embodiment, the amino acid sequence of the C2A domain is at least about 80%, about 85%, about 90%, about 95% or about 100% identical or similar to SEQ ID NO. 19. In an embodiment, the amino acid sequence of the FerA domain is at least about 80%, about 85%, about 90%, about 95% or about 100% identical or similar to SEQ ID NO. 20. In an embodiment, the amino acid sequence of the FerB is at least about 80%, about 85%, about 90%, about 95% or about 100% identical or similar to SEQ ID NO. 21. In an embodiment, the DysFN domain comprises DysFN-1, DysFN-2, or a combination thereof. In an embodiment, the amino acid sequence of the DysFN-1 is at least about 80%, about 85%, about 90%, about 95% or about 100% identical or similar to SEQ ID NO. 22. In an embodiment, the amino acid sequence of the DysFN-2 is at least about 80%, about 85%, about 90%, about 95% or about 100% identical or similar to SEQ ID NO. 23. In an embodiment, the amino acid sequence of the transmembrane domain is at least about 80%, about 85%, about 90%, about 95% or about 100% identical or similar to SEQ ID NO. 18.

SEQ ID NO. 19 (C2A):
MLRVIVESASNIPKTKFGKPDPIVSVIFKDEKKKTKKVDNELNPVWNEIL
EFDLRGIPLDFSSSLGIIVKDFETIGQNKLIGTATVALKDLTGDQSRSLP
YKLISLLNERGQDTGATIDLVIGYDPPSAPHPNDLS
SEQ ID NO. 20 (FerA):
LQTNIEALKSGIQGKIPANQLAELWLKLIDEVIEDTRYTLPLTEGKANVT
VLDTQIRK
SEQ ID NO. 21 (FerB):
WLDKLMQLTEEPQNSMPDIIIWMIRGEKRLAYARIPAHQVLYSTSGENAS
GKYCGKTQTIFLKYPQEKNNGP
SEQ ID NO. 22 (DysFN-1):
AVEKKFNSFAEGTFTVFAEMYENQALMFGKWGTSGLVGRHKFSDVTGKIK
LKREFFLP
SEQ ID NO. 23 (DysFN-2):
DPERSLLTEADAGHTEFTDEVYQNESRYPGGDWKPAEDTYTDANGDKAAS
PSELTCP

In an embodiment, any embodiment of the modified myoferlin protein of the present invention further comprises C2B, C2C, C2D, C2E domains, or a combination thereof. In an embodiment, the amino acid sequence of the C2B domain is at least about 80%, about 85%, about 90%, about 95% or about 100% identical or similar to SEQ ID NO. 24. In an embodiment, the amino acid sequence of the C2C domain is at least about 80%, about 85%, about 90%, about 95% or about 100% identical or similar to SEQ ID NO. 25. In an embodiment, the amino acid sequence of the C2D is at least about 80%, about 85%, about 90%, about 95% or about 100% identical or similar to SEQ ID NO. 26. In an embodiment, the amino acid sequence of the C2E is at least about 80%, about 85%, about 90%, about 95% or about 100% identical or similar to SEQ ID NO. 27.

SEQ ID NO. 24 (C2B):
PQDFQIRVRVIEGRQLSGNNIRPVVKVHVCGQTHRTRIKRGNNPFFDELF
FYNVNMTPSELMDEIISIRVYNSHSLRADCLMGEFKIDVGFVYDEPGHAV
MRKWLLLNDP
SEQ ID NO. 25 (C2C):
TFLLKIYRAEDIPQMDDAFSQTVKEIFGGNADKKNLVDPFVEVSFAGKKV
CTNIIEKNANPEWNQVVNLQIKFPSVCEKIKLTIYDWDRLTKNDVVGTTY
LHLSKIAASGGEVEDFSSSGTGAASYTVNTGETEVGFVPTFGPCYLNLYG
SPREYTGFPDPYDE
SEQ ID NO. 26 (C2D):
TPIVSCNFDRVYIYHLRCYVYQARNLLALDKDSFSDPYAHICFLHRSKTT
EIIHSTLNPTWDQTIIFDEVEIYGEPQTVLQNPPKVIMELFDNDQVGKDE
FLGRSIFSPVVKLNSEMDITPKLLWHPVMNGDKA
SEQ ID NO. 27 (C2E):
RNMKNFQMASITSPSLVVECGGERVESVVIKNLKKTPNFPSSVLFMKVFL
PKEELYMPPLVIKVIDHRQFGRKPVVG

In an embodiment, the modified myoferlin protein of the present invention does not comprise the C2A domain wherein the amino acid sequence of the C2A domain is at least about 80%, about 85%, about 90%, about 95% or about 100% identical or similar to SEQ ID NO. 19. In an embodiment, the modified myoferlin protein of the present invention does not comprise the C2B domain wherein the amino acid sequence of the C2B domain is at least about 80%, about 85%, about 90%, about 95% or about 100% identical or similar to SEQ ID NO. 24. In an embodiment, the modified myoferlin protein of the present invention does not comprise the C2C domain wherein the amino acid sequence of the C2C domain is at least about 80%, about 85%, about 90%, about 95% or about 100% identical or similar to SEQ ID NO. 25. In an embodiment, the modified myoferlin protein of the present invention does not comprise the C2D domain wherein the amino acid sequence of the C2D domain is at least about 80%, about 85%, about 90%, about 95% or about 100% identical or similar to SEQ ID NO. 26. In an embodiment, the modified myoferlin protein of the present invention does not comprise the C2E domain wherein the amino acid sequence of the C2E domain is at least about 80%, about 85%, about 90%, about 95% or about 100% identical or similar to SEQ ID NO. 27. In an embodiment, the modified myoferlin protein of the present invention does not comprise the C2F domain wherein the amino acid sequence of the C2F domain is at least about 80%, about 85%, about 90%, about 95% or about 100% identical or similar to SEQ ID NO. 16. In an embodiment, the modified myoferlin protein of the present invention does not comprise the C2G domain wherein the amino acid sequence of the C2G domain is at least about 80%, about 85%, about 90%, about 95% or about 100% identical or similar to SEQ ID NO. 17. In an embodiment, the modified myoferlin protein of the present invention does not comprise the FerA domain wherein the amino acid sequence of the FerA domain is at least about 80%, about 85%, about 90%, about 95% or about 100% identical or similar to SEQ ID NO. 20. In an embodiment, the modified myoferlin protein of the present invention does not comprise the FerB domain wherein the amino acid sequence of the FerB domain is at least about 80%, about 85%, about 90%, about 95% or about 100% identical or similar to SEQ ID NO. 21. In an embodiment, the modified myoferlin protein of the present invention does not comprise the DysFN-1 domain wherein the amino acid sequence of the DysFN-1 domain is at least about 80%, about 85%, about 90%, about 95% or about 100% identical or similar to SEQ ID NO. 22. In an embodiment, the modified myoferlin protein of the present invention does not comprise the DysFN-2 domain wherein the amino acid sequence of the DysFN-2 domain is at least about 80%, about 85%, about 90%, about 95% or about 100% identical or similar to SEQ ID NO. 23. In an embodiment, the modified myoferlin protein of the present invention does not comprise the transmembrane domain wherein the amino acid sequence of the transmembrane domain is at least about 80%, about 85%, about 90%, about 95% or about 100% identical or similar to SEQ ID NO. 18.

In an embodiment, the modified myoferlin protein of the present invention comprises an amino acid sequence at least about 80%, about 85%, about 90%, about 95% or about 100% identical or similar to SEQ ID NO. 28.

SEQ ID NO. 28 (C2F-C2G):
MVPAPPRQFRELPDSVPQECTVRIYIVRGLELQPQDNNGLCDPYIKITLG
KKVIEDRDHYIPNTLNPVFGRMYELSCYLPQEKDLKISVYDYDTFTRDEK
VGETIIDLENRFLSRFGSHCGIPEEYCVSGVNTWRDQLRPTQLLQNVARF
KGFPQPILSEDGSRIRYGGRDYSLDEFEANKILHQHLGAPEERLALHILR
TQGLVPEHVETRTLHSTFQPNISQGKLQMWVDVFPKSLGPPGPPFNITPR
KAKKYYLRVIIWNTKDVILDEKSITGEEMSDIYVKGWVPGNEENKQKTDV
HYRSLDGEGNFNWRFVFPFDYLPAEQLCIVAKKEHFWSIDQTEFRIPPRL
IIQIWDNDKFSLDDYLGFLELDLRHTIIPAKSPEKCRLDMIPDLKAMNPL
KAKTASLFEQKSMKGWWPCYAEKDGARVMAGKVEMTLEILNEKEADERPA
GKGRDEPNMNPKLDLPNRPETSFLWFTNPCKTMKFIVWRRFKWVIIGLLF
LLILLLFVAVLLYSLPNYLSMKIVKPNVYPYDVPDYA

In an embodiment, any embodiment of the modified myoferlin protein of the present invention is in the form of a polynucleotide encoding said modified myoferlin protein. In an embodiment, any embodiment of the modified myoferlin protein of the present invention comprises a polynucleotide encoding a C2A domain, a C2B domain, a C2C domain, a C2D domain, a C2E domain, a C2F domain, a C2G domain, a FerA domain, a FerB domain, a DysFN-1 domain, a DysFN-2 domain, a transmembrane domain, or a combination thereof. In an embodiment, the C2A domain is encoded by a polynucleotide comprising a nucleotide sequence at least about 80%, about 85%, about 90%, about 95% or about 100% identical to SEQ ID NO. 29. In an embodiment, the C2B domain is encoded by a polynucleotide comprising a nucleotide sequence at least about 80%, about 85%, about 90%, about 95% or about 100% identical to SEQ ID NO. 30. In an embodiment, the C2C domain is encoded by a polynucleotide comprising a nucleotide sequence at least about 80%, about 85%, about 90%, about 95% or about 100% identical to SEQ ID NO. 31. In an embodiment, the C2D domain is encoded by a polynucleotide comprising a nucleotide sequence at least about 80%, about 85%, about 90%, about 95% or about 100% identical to SEQ ID NO. 32. In an embodiment, the C2E domain is encoded by a polynucleotide comprising a nucleotide sequence at least about 80%, about 85%, about 90%, about 95% or about 100% identical to SEQ ID NO. 33. In an embodiment, the C2F domain is encoded by a polynucleotide comprising a nucleotide sequence at least about 80%, about 85%, about 90%, about 95% or about 100% identical to SEQ ID NO. 34. In an embodiment, the C2G domain is encoded by a polynucleotide comprising a nucleotide sequence at least about 80%, about 85%, about 90%, about 95% or about 100% identical to SEQ ID NO. 35. In an embodiment, the FerA domain is encoded by a polynucleotide comprising a nucleotide sequence at least about 80%, about 85%, about 90%, about 95% or about 100% identical to SEQ ID NO. 36. In an embodiment, the FerB domain is encoded by a polynucleotide comprising a nucleotide sequence at least about 80%, about 85%, about 90%, about 95% or about 100% identical to SEQ ID NO. 37. In an embodiment, the DysFN-1 domain is encoded by a polynucleotide comprising a nucleotide sequence at least about 80%, about 85%, about 90%, about 95% or about 100% identical to SEQ ID NO. 38. In an embodiment, the DysFN-2 domain is encoded by a polynucleotide comprising a nucleotide sequence at least about 80%, about 85%, about 90%, about 95% or about 100% identical to SEQ ID NO. 39. In an embodiment, the transmembrane domain is encoded by a polynucleotide comprising a nucleotide sequence at least about 80%, about 85%, about 90%, about 95% or about 100% identical to SEQ ID NO. 40.

SEQ ID NO. 29 (nt C2A):
ATGCTGCGAGTGATTGTGGAATCTGCCAGCAATATCCCTAAAACGAAATTTGGC
AAGCCGGATCCTATTGTTTCTGTCATTTTTAAGGATGAGAAAAAGAAAACAAAG
AAAGTTGATAATGAATTGAACCCTGTCTGGAATGAGATTTTGGAGTTTGACTTGA
GGGGTATACCACTGGACTTTTCATCTTCCCTTGGGATTATTGTGAAAGATTTTGA
GACAATTGGACAAAATAAATTAATTGGCACGGCGACTGTAGCCCTGAAGGACCT
GACTGGTGACCAGAGCAGATCCCTGCCGTACAAGCTGATCTCCCTGCTAAATGA
AAGAGGGCAAGATACTGGGGCCACCATTGACTTGGTGATCGGCTATGATCCGCC
TTCTGCTCCACATCCAAATGACCTGAGC
SEQ ID NO. 30 (nt C2B):
CCACAGGACTTCCAGATCCGCGTCCGAGTGATTGAGGGCCGACAGTTAAGTGGC
AACAACATAAGGCCTGTGGTCAAAGTTCACGTCTGTGGCCAGACACACCGAACA
AGAATCAAGAGAGGAAACAACCCTTTTTTTGATGAGTTGTTTTTCTACAATGTCA
ACATGACCCCTTCTGAATTGATGGATGAGATCATCAGCATCCGGGTTTATAATTC
TCACTCTCTGCGGGCAGATTGTCTGATGGGGGAATTTAAGATTGATGTTGGATTT
GTTTATGATGAACCTGGCCATGCTGTCATGAGAAAGTGGCTTCTTCTCAATGACC
CG
SEQ ID NO. 31 (nt C2C):
ACCTTCTTGCTGAAAATCTACCGAGCTGAGGACATCCCCCAGATGGATGATGCC
TTCTCACAGACAGTAAAGGAAATATTTGGAGGCAATGCAGATAAGAAAAATCTC
GTGGATCCTTTTGTAGAAGTTTCCTTTGCTGGAAAAAAGGTTTGTACAAACATAA
TTGAGAAGAATGCAAACCCAGAGTGGAATCAGGTCGTCAATCTTCAGATCAAGT
TTCCTTCAGTGTGTGAAAAAATAAAACTAACAATATATGACTGGGACCGTCTTA
CTAAAAATGATGTAGTTGGAACAACATATCTACACCTCTCTAAAATTGCTGCCTC
TGGTGGGGAAGTGGAAGATTTCTCATCTTCGGGAACTGGGGCTGCATCATATAC
AGTAAACACAGGAGAAACAGAGGTAGGCTTTGTTCCAACGTTTGGACCTTGTTA
CCTGAATCTTTATGGAAGCCCCAGAGAGTACACGGGATTCCCAGACCCCTATGA
TGAG
SEQ ID NO. 32 (nt C2D):
ACCCCCATTGTTTCCTGCAATTTTGACAGAGTCTACATCTACCATCTGCGCTGCT
ATGTCTATCAAGCCAGAAACCTCTTGGCTTTAGATAAGGATAGCTTTTCAGATCC
ATATGCTCATATCTGTTTCCTCCATCGGAGCAAAACCACTGAGATCATCCATTCA
ACCCTGAATCCCACGTGGGACCAAACAATTATATTCGATGAAGTTGAAATCTAT
GGGGAACCCCAAACAGTTCTACAGAATCCACCCAAAGTTATCATGGAACTTTTT
GACAATGACCAAGTGGGCAAAGATGAATTTTTAGGACGAAGCATTTTCTCTCCT
GTGGTGAAACTGAACTCAGAAATGGACATCACACCCAAACTTCTCTGGCACCCA
GTAATGAATGGAGACAAAGCC
SEQ ID NO. 33 (nt C2E):
AGAAATATGAAAAACTTCCAGATGGCTTCTATCACATCCCCCAGTCTTGTTGTGG
AGTGTGGAGGAGAAAGGGTGGAATCGGTGGTGATCAAAAACCTTAAGAAGACA
CCCAACTTTCCAAGTTCTGTTCTCTTCATGAAAGTGTTCTTGCCCAAGGAGGAAT
TGTACATGCCCCCACTGGTGATCAAGGTCATCGACCACAGGCAGTTTGGGCGGA
AGCCTGTCGTCGGC
SEQ ID NO. 34 (nt C2F):
CAGTTTCGGGAATTACCTGACAGCGTCCCACAGGAATGCACGGTTAGGATTTAC
ATTGTTCGAGGCTTAGAGCTCCAGCCCCAGGACAACAATGGCCTGTGTGACCCT
TACATAAAAATAACACTGGGCAAAAAAGTCATTGAAGACCGAGATCACTACATT
CCCAACACTCTCAACCCAGTCTTTGGCAGGATGTACGAACTGAGCTGCTACTTAC
CTCAAGAAAAAGACCTGAAAATTTCTGTCTATGATTATGACACCTTTACCCGGG
ATGAAAAAGTAGGAGAGACAATTATTGATCTGGAAAACCGATTCCTTTCCCGCT
TTGGGTCCCACTGCGGCATACCAGAGGAGTACTGTGTTTCTGGAGTC
SEQ ID NO. 35 (nt C2G):
CCTTTCAACATCACACCCCGGAAAGCCAAGAAATACTACCTGCGTGTGATCATC
TGGAACACCAAGGATGTTATCTTGGATGAGAAAAGCATCACAGGAGAGGAAAT
GAGTGACATCTACGTCAAAGGCTGGGTTCCTGGCAATGAAGAAAACAAACAGA
AAACAGATGTCCATTACAGATCTTTGGATGGTGAAGGGAATTTTAACTGGCGAT
TTGTTTTCCCGTTTGACTACCTTCCAGCCGAACAACTCTGTATCGTTGCGAAAAA
AGAGCATTTCTGGAGTATTGACCAAACGGAATTTCGAATCCCACCCAGGCTGAT
CATTCAGATATGGGACAATGACAAGTTTTCTCTGGATGACTACTTGGGTTTCCTA
GAACTTGACTTGCGTCAC
SEQ ID NO. 36 (nt FerA):
CTGCAAACAAATATAGAAGCTCTAAAATCAGGGATACAAGGTAAAATTCCTGCA
AACCAGCTGGCTGAATTGTGGCTGAAGCTGATAGATGAAGTTATAGAAGACACG
AGATACACGTTGCCTCTCACAGAAGGAAAAGCCAACGTCACAGTTCTCGATACT
CAGATCCGAAAG
SEQ ID NO. 37 (nt FerB):
TGGCTTGATAAATTAATGCAGCTGACTGAAGAGCCACAGAACAGCATGCCTGAC
ATCATCATCTGGATGATCCGGGGAGAGAAGAGACTGGCCTATGCACGAATTCCC
GCACATCAGGTCTTGTACTCCACCAGTGGTGAGAATGCATCTGGAAAATACTGT
GGGAAAACCCAAACCATCTTTCTGAAGTATCCACAGGAGAAAAACAACGGGCC
A
SEQ ID NO. 38 (nt DysFN-1):
GCTGTGGAGAAGAAGTTTAACAGCTTCGCAGAAGGAACTTTCACCGTCTTTGCT
GAAATGTATGAAAATCAAGCTCTCATGTTTGGAAAATGGGGTACTTCTGGATTA
GTAGGACGTCATAAGTTTTCTGATGTCACAGGAAAAATAAAACTCAAGAGGGAA
TTTTTTCTGCCT
SEQ ID NO. 39 (nt DysFN-2):
GATCCTGAAAGAAGCTTGCTGACTGAGGCAGATGCAGGTCACACGGAGTTCACT
GATGAAGTCTACCAGAACGAGAGCCGCTACCCCGGGGGCGACTGGAAGCCGGC
CGAGGACACCTACACGGATGCGAACGGCGATAAAGCAGCATCACCCAGCGAGT
TGACTTGTCCT
SEQ ID NO. 40 (nt TM):
CCGGACCTCAAAGCCATGAACCCCCTTAAAGCCAAGACAGCCTCCCTCTTTGAG
CAGAAGTCCATGAAAGGATGGTGGCCATGCTACGCAGAGAAAGATGGCGCCCG
CGTAATGGCTGGGAAAGTGGAGATGACATTGGAAATCCTCAACGAGAAGGAGG
CCGACGAGAGGCCAGCCGGGAAGGGGCGGGACGAACCCAACATGAACCCCAAG
CTGGACTTACCAAATCGACCAGAAACCTCCTTCCTCTGGTTCACCAACCCATGCA
AGACCATGAAGTTCATCGTGTGGCGCCGCTTTAAGTGGGTCATCATCGGCTTGCT
GTTCCTGCTTATCCTGCTGCTCTTCGTGGCCGTG

In an embodiment, the modified myoferlin protein of the present invention further comprises a connexin 43 protein, wherein said dual protein comprising the modified myoferlin protein and the connexin 43 protein is capable of substantially enhancing the efficacy and delivery of the RNA or nucleic acid payloads. In an embodiment, the connexin protein comprises a S368A mutation. In an embodiment, the connexin protein of the present invention comprises an amino acid sequence at least about 80%, about 85%, about 90%, about 95% or about 100% identical or similar to SEQ ID NO. 41.

SEQ ID NO. 41 (connexin 43):
MATTMGDWSALGKLLDKVQAYSTAGGKVWLSVLFIFRILLLGTAVESAWG
DEQSAFRCNTQQPGCENVCYDKSFPISHVRFWVLQIIFVSVPTLLYLAHV
FYVMRKEEKLNKKEEELKVAQTDGVNVDMHLKQIEIKKFKYGIEEHGKVK
MRGGLLRTYIISILFKSIFEVAFLLIQWYIYGFSLSAVYTCKRDPCPHQV
DCFLSRPTEKTIFIIFMLVVSLVSLALNIIELFYVFFKGVKDRVKGKSDP
YHATSGALSPAKDCGSQKYAYFNGCSSPTAPLSPMSPPGYKLVTGDRNNS
SCRNYNKQASEQNWANYSAEQNRMGQAGSTISNSHAQPFDFPDDNQNSKK
LAAGHELQPLAIVDQRPSSRAASRASSRPRPDDLEI

In an embodiment, the dual protein comprising a modified myoferlin protein fused with a connexin 43 protein is encoded by a polynucleotide wherein the nucleotide sequence of the polynucleotide is at least about 80%, about 85%, about 90%, about 95% or about 100% identical to SEQ ID NO. 42.

SEQ ID NO. 42:
ATGGTGCCAGCCCCTCCCAGACAGTTTCGGGAATTACCTGACAGCGTCCC
ACAGGAATGCACGGTTAGGATTTACATTGTTCGAGGCTTAGAGCTCCAGCCCCA
GGACAACAATGGCCTGTGTGACCCTTACATAAAAATAACACTGGGCAAAAAAGT
CATTGAAGACCGAGATCACTACATTCCCAACACTCTCAACCCAGTCTTTGGCAG
GATGTACGAACTGAGCTGCTACTTACCTCAAGAAAAAGACCTGAAAATTTCTGT
CTATGATTATGACACCTTTACCCGGGATGAAAAAGTAGGAGAGACAATTATTGA
TCTGGAAAACCGATTCCTTTCCCGCTTTGGGTCCCACTGCGGCATACCAGAGGAG
TACTGTGTTTCTGGAGTCAATACCTGGCGAGATCAACTGAGACCAACACAGCTG
CTTCAAAATGTCGCCAGATTCAAAGGCTTCCCACAACCCATCCTTTCCGAAGATG
GGAGTAGAATCAGATATGGAGGACGAGACTACAGCTTGGATGAATTTGAAGCC
AACAAAATCCTGCACCAGCACCTCGGGGCCCCTGAAGAGCGGCTTGCTCTTCAC
ATCCTCAGGACTCAGGGGCTGGTCCCTGAGCACGTGGAAACAAGGACTTTGCAC
AGCACCTTCCAGCCCAACATTTCCCAGGGAAAACTTCAGATGTGGGTGGATGTT
TTCCCCAAGAGTTTGGGGCCACCAGGCCCTCCTTTCAACATCACACCCCGGAAA
GCCAAGAAATACTACCTGCGTGTGATCATCTGGAACACCAAGGATGTTATCTTG
GATGAGAAAAGCATCACAGGAGAGGAAATGAGTGACATCTACGTCAAAGGCTG
GGTTCCTGGCAATGAAGAAAACAAACAGAAAACAGATGTCCATTACAGATCTTT
GGATGGTGAAGGGAATTTTAACTGGCGATTTGTTTTCCCGTTTGACTACCTTCCA
GCCGAACAACTCTGTATCGTTGCGAAAAAAGAGCATTTCTGGAGTATTGACCAA
ACGGAATTTCGAATCCCACCCAGGCTGATCATTCAGATATGGGACAATGACAAG
TTTTCTCTGGATGACTACTTGGGTTTCCTAGAACTTGACTTGCGTCACACGATCA
TTCCTGCAAAATCACCAGAGAAATGCAGGTTGGACATGATTCCGGACCTCAAAG
CCATGAACCCCCTTAAAGCCAAGACAGCCTCCCTCTTTGAGCAGAAGTCCATGA
AAGGATGGTGGCCATGCTACGCAGAGAAAGATGGCGCCCGCGTAATGGCTGGG
AAAGTGGAGATGACATTGGAAATCCTCAACGAGAAGGAGGCCGACGAGAGGCC
AGCCGGGAAGGGGCGGGACGAACCCAACATGAACCCCAAGCTGGACTTACCAA
ATCGACCAGAAACCTCCTTCCTCTGGTTCACCAACCCATGCAAGACCATGAAGTT
CATCGTGTGGCGCCGCTTTAAGTGGGTCATCATCGGCTTGCTGTTCCTGCTTATC
CTGCTGCTCTTCGTGGCCGTGCTCCTCTACTCTTTGCCGAACTATTTGTCAATGAA
GATTGTAAAGCCAAATGTGTACCCATACGACGTCCCAGACTACGCTTAGGCCCC
TCTCCCTCCCCCCCCCCTAACGTTACTGGCCGAAGCCGCTTGGAATAAGGCCGGT
GTGCGTTTGTCTATATGTTATTTTCCACCATATTGCCGTCTTTTGGCAATGTGAGG
GCCCGGAAACCTGGCCCTGTCTTCTTGACGAGCATTCCTAGGGGTCTTTCCCCTC
TCGCCAAAGGAATGCAAGGTCTGTTGAATGTCGTGAAGGAAGCAGTTCCTCTGG
AAGCTTCTTGAAGACAAACAACGTCTGTAGCGACCCTTTGCAGGCAGCGGAACC
CCCCACCTGGCGACAGGTGCCTCTGCGGCCAAAAGCCACGTGTATAAGATACAC
CTGCAAAGGCGGCACAACCCCAGTGCCACGTTGTGAGTTGGATAGTTGTGGAAA
GAGTCAAATGGCTCTCCTCAAGCGTATTCAACAAGGGGCTGAAGGATGCCCAGA
AGGTACCCCATTGTATGGGATCTGATCTGGGGCCTCGGTGCACATGCTTTACATG
TGTTTAGTCGAGGTTAAAAAAACGTCTAGGCCCCCCGAACCACGGGGACGTGGT
TTTCCTTTGAAAAACACGATGATAATATGGCCACAACCATGGGTGACTGGAGCG
CCTTAGGCAAACTCCTTGACAAGGTTCAAGCCTACTCAACTGCTGGAGGGAAGG
TGTGGCTGTCAGTACTTTTCATTTTCCGAATCCTGCTGCTGGGGACAGCGGTTGA
GTCAGCCTGGGGAGATGAGCAGTCTGCCTTTCGTTGTAACACTCAGCAACCTGG
TTGTGAAAATGTCTGCTATGACAAGTCTTTCCCAATCTCTCATGTGCGCTTCTGG
GTCCTGCAGATCATATTTGTGTCTGTACCCACACTCTTGTACCTGGCTCATGTGTT
CTATGTGATGCGAAAGGAAGAGAAACTGAACAAGAAAGAGGAAGAACTCAAGG
TTGCCCAAACTGATGGTGTCAATGTGGACATGCACTTGAAGCAGATTGAGATAA
AGAAGTTCAAGTACGGTATTGAAGAGCATGGTAAGGTGAAAATGCGAGGGGGG
TTGCTGCGAACCTACATCATCAGTATCCTCTTCAAGTCTATCTTTGAGGTGGCCT
TCTTGCTGATCCAGTGGTACATCTATGGATTCAGCTTGAGTGCTGTTTACACTTG
CAAAAGAGATCCCTGCCCACATCAGGTGGACTGTTTCCTCTCTCGCCCCACGGA
GAAAACCATCTTCATCATCTTCATGCTGGTGGTGTCCTTGGTGTCCCTGGCCTTG
AATATCATTGAACTCTTCTATGTTTTCTTCAAGGGCGTTAAGGATCGGGTTAAGG
GAAAGAGCGACCCTTACCATGCGACCAGTGGTGCGCTGAGCCCTGCCAAAGACT
GTGGGTCTCAAAAATATGCTTATTTCAATGGCTGCTCCTCACCAACCGCTCCCCT
CTCGCCTATGTCTCCTCCTGGGTACAAGCTGGTTACTGGCGACAGAAACAATTCT
TCTTGCCGCAATTACAACAAGCAAGCAAGTGAGCAAAACTGGGCTAATTACAGT
GCAGAACAAAATCGAATGGGGCAGGCGGGAAGCACCATCTCTAACTCCCATGC
ACAGCCTTTTGATTTCCCCGATGATAACCAGAATTCAAAAAAACTAGCTGCTGG
ACATGAATTACAGCCACTAGCCATTGTGGACCAGCGACCTTCAAGCAGAGCCGC
CAGTCGTGCCAGCAGCAGACCTCGGCCTGATGACCTGGAGATCTGA

In an embodiment, any embodiment of the nanoparticle encapsulating polynucleotide of the present invention upregulates the expression of the ELN gene in a subject by at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 100%, about 150%, about 200%, about 250%, about 300%, about 350%, about 500%, about 750% or about 1000% compared to the level of the expression of the ELN gene in the subject prior to administration of the polynucleotide of the present invention to the subject. In an embodiment, any embodiment of the nanoparticle encapsulating the polynucleotide of the present invention upregulates the expression of the ELN gene in a subject to restore the total amount of elastin in the subject to the same total amount in the subject as about 1 year, about 2 years, about 3 years, about 5 years, about 10 years, about 12 years, about 14 years, about 16 years, about 18 years, about 20 years, about 25 years, about 30 years, about 35 years, about 40 years about 50 years, about 60 years, about 70 years, about 80 years ago. In an embodiment, any embodiment of the nanoparticle encapsulating the polynucleotide of the present invention upregulates the expression of the ELN gene in a subject to restore the total amount of elastin in the subject to the same total amount of elastin in a normal healthy human of an age younger than the subject by about 1 year, about 2 years, about 3 years, about 5 years, about 10 years, about 12 years, about 14 years, about 16 years, about 18 years, about 20 years, about 25 years, about 30 years, about 35 years, about 40 years about 50 years, about 60 years, about 70 years, about 80 years ago.

In an embodiment, any embodiment of the nanoparticle encapsulating the polynucleotide of the present invention restores the expression of the ELN gene in a subject to at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% of the normal ELN gene expression level of the subject when healthy. In an embodiment, any embodiment of the nanoparticle encapsulating the polynucleotide of the present invention restores the expression of the ELN gene to at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% of normal ELN expression level of an average healthy human being. In an embodiment, any embodiment of the nanoparticle encapsulating the polynucleotide of the present invention restores the expression of the ELN gene to at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% of normal ELN expression level of an average healthy human being having the same biometrics of the subject such as age, sex, height, weight etc., or a combination thereof.

In an embodiment, any embodiment of the nanoparticle encapsulating the polynucleotide of the present invention increases the ratio of non-mutated: mutated ELN gene transcript in a subject. In an embodiment, any embodiment of the nanoparticle encapsulating the polynucleotide of the present invention increases the ratio of non-mutated: mutated ELN gene transcript by at least around 5%, around 10%, around 15%, around 20%, around 25%, around 30%, around 40%, around 50%, around 60%, around 70%, around 80%, around 90%, around 100%, around 150%, around 200%, around 250%, around 300%, around 350%, around 400%, around 450%, or around 500%. In an embodiment, the ELN gene expression in the subject comprises disease-associated deletions or mutations. In an embodiment, at least about 5%, about 10%, about 15%, about 20%, about 25%, about 30%, about 35%, about 40%, about 45%, about 50%, about 54%, about 60%, about 65%, about 70%, about 75%, about 80%, about 85%, about 90%, about 95%, or about 100% ELN gene expression in the subject comprises disease-associated deletions or mutations. In an embodiment, the disease is an ELN-associated disease. In an embodiment, the subject is diagnosed with an ELN-associated disease. In an embodiment, the ELN-associated disease comprises supravalvular aortic stenosis (SVAS), autosomal dominant cutis laxa, Marfan syndrome, alpha1-antitrypsin deficiency-related emphysema, atherosclerosis, Buschke-Ollendorff syndrome, Menkes syndrome, pseudoxanthoma elasticum, and Williams syndrome. In an embodiment, the subject is deficient in elastin. The deficiency in elastin of a subject can be a result of aging, wounding, or any diseases leading to such deficiency. In an embodiment, the subject is in need for cosmetic treatment such as scar revision or correction of burned, saggy or wrinkle skin. In an embodiment, the subject comprises a cell, a tissue, or an organ of a mammal such as a human or a mouse.

The present invention also provides a method of altering the ELN gene expression in a subject. In an embodiment, the method of altering the ELN gene expression in a subject comprises upregulating the ELN gene expression, restoring the ELN gene expression, or increasing the ratio of non-mutated: mutated ELN gene transcript in a subject. In an embodiment, the method of altering the ELN gene expression of the present invention comprises the step of administering a therapeutically effective amount of any embodiment of the pharmaceutical composition comprising the polynucleotide of the present invention. In an embodiment, the method of altering the ELN gene expression in a subject of the present invention upregulates the expression of the ELN gene in a subject by at least about 10%, about 20%, about 30%, about 40%, about 50%, about 60%, about 70%, about 80%, about 90%, about 100%, about 150%, about 200%, about 250%, about 300%, about 350%, about 500%, about 750% or about 1000% compared to the level of the expression of the ELN gene in the subject prior to administration of the polynucleotide of the present invention to the subject. In an embodiment, the method of altering the ELN gene expression in a subject of the present invention upregulates the expression of the ELN gene in a subject to restore the total amount of Elastin in the subject to the same total amount as about 1 year, about 2 years, about 3 years, about 5 years, about 10 years, about 12 years, about 14 years, about 16 years, about 18 years, about 20 years, about 25 years, about 30 years, about 35 years, about 40 years about 50 years, about 60 years, about 70 years, about 80 years ago. In an embodiment, the method of altering the ELN gene expression in a subject of the present invention upregulates the expression of the ELN gene in a subject to restore the total amount of elastin in the subject to the same total amount of elastin in a normal healthy human of an age younger than the subject by about 1 year, about 2 years, about 3 years, about 5 years, about 10 years, about 12 years, about 14 years, about 16 years, about 18 years, about 20 years, about 25 years, about 30 years, about 35 years, about 40 years about 50 years, about 60 years, about 70 years, about 80 years ago.

In an embodiment, the method of altering the ELN gene expression in a subject restores the expression of the ELN gene in a subject to at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% of the normal ELN gene expression level of the subject when healthy. In an embodiment, the method of altering the ELN gene expression in a subject restores the expression of the ELN gene to at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% of normal ELN expression level of an average healthy human being. In an embodiment, the method of altering the ELN gene expression in a subject restores the expression of the ELN gene to at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% of normal ELN expression level of an average healthy human being having the same biometrics of the subject such as age, sex, height, weight etc., or a combination thereof. In an embodiment, the method of altering the ELN gene expression in a subject increases the ratio of non-mutated: mutated ELN gene transcript in a subject. In an embodiment, the method of altering the ELN gene expression in a subject increases the ratio of non-mutated: mutated ELN gene transcript by at least about 5%, about 10%, about 15%, about 20%, about 25%, about 30%, about 40%, about 50%, about 60%, about 70%, about 80%, about 90%, about 100%, about 150%, about 200%, about 250%, about 300%, about 350%, about 400%, about 450%, or about 500%. In an embodiment, the ELN gene expression in the subject comprises disease-associated deletions or mutations. In an embodiment, at least about 5%, about 10%, about 15%, about 20%, about 25%, about 30%, about 35%, about 40%, about 45%, about 50%, about 54%, about 60%, about 65%, about 70%, about 75%, about 80%, about 85%, about 90%, about 95%, or about 100% ELN gene expression in the subject comprises disease-associated deletions or mutations. In an embodiment, the disease is an ELN-associated disease. In an embodiment, the subject is diagnosed with an ELN-associated disease. In an embodiment, the ELN-associated disease comprises supravalvular aortic stenosis (SVAS), autosomal dominant cutis laxa, Marfan syndrome, alpha1-antitrypsin deficiency-related emphysema, atherosclerosis, Buschke-Ollendorff syndrome, Menkes syndrome, pseudoxanthoma elasticum, and Williams syndrome. In an embodiment, the subject is deficient with elastin. The deficiency in elastin of a subject can be a result of aging, wounding, or any diseases leading to such deficiency. In an embodiment, the subject is in need for cosmetic treatment such as scar revision or correction of burned, saggy or wrinkle skin. In an embodiment, the subject comprises a cell, a tissue, or an organ of a mammal such as a human or a mouse.

The present invention also provides a method of treatment of an ELN-associated disease of a subject comprising a step of altering the ELN gene expression in a subject by administration of a therapeutically effective amount of any embodiment of the pharmaceutical composition comprising the polynucleotide of the present invention to the subject. In an embodiment, the subject is diagnosed with an ELN-associated disease. In an embodiment, the ELN-associated disease comprises supravalvular aortic stenosis (SVAS), autosomal dominant cutis laxa, Marfan syndrome, alpha1-antitrypsin deficiency-related emphysema, atherosclerosis, Buschke-Ollendorff syndrome, Menkes syndrome, pseudoxanthoma elasticum, and Williams syndrome. In an embodiment, the subject is deficient in elastin. The deficiency in elastin of a subject can be a result of aging, wounding, or any diseases leading to such deficiency. In an embodiment, the subject is in need for cosmetic treatment such as scar revision or correction of burned, saggy or wrinkle skin. In an embodiment, the subject comprises a cell, a tissue, or an organ of a mammal such as a human or a mouse.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal, intramuscular injection, intrathecal, intraventricular, intracerebral, or intracerebroventricular administration. In an embodiment, the administration may be through microneedle administration, wherein the microneedle administration can comprise either passive method or active method. In an embodiment, the microneedle administration comprises a solid microneedle patch, a coated microneedle patch, a dissolving microneedle patch, a hydrogel microneedle patch, a hallow microneedle patch, a separable microneedle patch, or a swellable microneedle patch. The route and site of administration may be chosen to enhance delivery or targeting of the disrupting agent comprising a site-specific targeting moiety to a particular location.

It is to be understood that both the foregoing general description and detailed description are exemplary and explanatory only and are not restrictive of the invention, as claimed. In general, the terms used in the disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

### EXAMPLES

### Materials and Methods

### Generation of Stable Elastin-Exosome Expressing HEK293 Cell Lines.

Several stable Elastin mRNA packaged exosome producing cell lines were developed. The cell lines were generated by transfecting replicates of HEK293 cells (Table 1) with the following plasmid cocktails (Table 2). Following transfection cells were treated with varying concentrations of triple drug (Puromycin/Blastocydin/G418-Neomycin, Table 3) and expanded. The surviving cultures of triple drug resistant cell lines are used to continuously generate Elastin packaged exosomes.

The stable cell lines generated will be expanded and serve as our parent ELN RNA-EV cell lines.

### Cell Culture

Human embryonic kidney 293T (HEK-293T) cells were cultured in Dulbecco's Modified Eagle Medium supplemented with 10% Fetal Bovine Serum and 1% L-Glutamine and grown in a humidified incubator at 37 °C with 5% CO₂. Cells were cultured into T25 flasks from frozen cell pellets and expanded into T75 flasks. Cell cultures were normally passaged to keep healthy growth after reaching 80-90% confluency and under constant drug selection (Table 3).

### RNA Isolation and Reverse Transcription

The transfected cells were collected after lysis buffer treatment then centrifuged at 300×g for 5 minutes. The RNA of the cells was harvested using the Qiagen RNeasy Mini Kit. The RNA from transfected cells was checked the purity by using Nanodrop and then reverse transcribed into cDNA using the LunaScript® RT SuperMix Kit and associated protocol.

qRT-PCR

The Promega GoTaq qPCR Sybr green was used for master mix preparation. For one sample, this required 7 μL of Sybr green master mix with 1.4 μL of each forward and reverse primer, 3.08 μL of RNase free water and 0.5 μL of cDNA (≤100 ng/rxn). The cycling conditions for the synthesis of qRT-PCR were initial denaturation at 95 °C for 10 minutes, denaturation at 95 °C for 15 seconds, annealing at 60 °C for 15 seconds, extension at 72 °C for 30 seconds (40 cycles), and a final extension step at 72 °C for 10 minutes. These were run using both the Qaigen Rotor-gene PCR cycler. Housekeeping beta actin mRNA was used as internal control for comparison of relative RNA expression. Relative gene expression was evaluated by using 2(−ΔΔCT) method. Primers are shown in Table 4.

TABLE 1
Example 24 well plates. Transfect treatments in quadruplicate
Plate A

| | Treatment (Plasmid DNA transfected) | | | | Cells alone | |
|---|---|---|---|---|---|---|
| Conc-1 | (1) Control | (2) | (3) | (4) | (5) | (6) |
| Conc-2 | (1) Control | (2) | (3) | (4) | (5) | (6) |
| Conc-3 | (1) Control | (2) | (3) | (4) | (5) | (6) |
| Conc-4 | (1) Control | (2) | (3) | (4) | (5) | (6) |

TABLE 2
Treatment Candidate stable ELN EV cell lines to generate by co-transfecting HEK2923
producer cells followed by triple/quadruple drug antibiotic selection.

| Treatment | Plasmid number in bold, 4 Plasmids to transfect into HEK producer |
|---|---|
| (drug selection) | cells/ ng for cocktail |
| (1) ELN-CD63-U | (1) p25 pRP[Exp]-Bsd-EF1A > {CD63-L7ae} (RNA packer)/150 ng |
| (Bsd) | (2) p30 pVB230615-1543puy_{—}Myo_{—}Cnx43_{—}Neomycin/150 ng |
| | (3) p16 pLV[Exp]-Hygro-EF1A > {ELN1-CD-U1a(human opt)}- |
| | 2xC/Dbox-2xU1a_RBS/25 ng |
| (2) ELN-CD63-L | (1) p26 pRP[Exp]-Bsd-EF1A > {CD63-U1a} (RNA packer)/ 150 ng |
| (Bsd) | (2) p30 pVB230615-1543puy_{—}Myo_{—}Cnx43_{—}Neomycin/150 ng |
| | (3) p16 pLV[Exp]-Hygro-EF1A > {ELN1-CD-U1a(human opt)}- |
| | 2xC/Dbox-2xU1a_RBS/25 ng |
| (3) ELN-CD63-U | (1) p27 pLV[Exp]-Puro-CMV + intron > {PTGFRN-U1a}(RNA packer)/ |
| (puro) | 150 ng |
| | (2) p30 pVB230615-1543puy_Myo_Cnx43_Neomycin/150 ng |
| | (3) p16 pLV[Exp]-Hygro-EF1A > {ELN1-CD-U1a(human opt)}- |
| | 2xC/Dbox-2xU1a_RBS/25 ng |
| (4) ELN-CD63-L | (1) p28 pLV[Exp]-Puro-CMV + intron > {CD81-U1a}/150 ng |
| (puro) | (2) p30 pVB230615-1543puy_{—}Myo_{—}Cnx43_{—}Neomycin/150 ng |
| | (3) p16 pLV[Exp]-Hygro-EF1A > {ELN1-CD-U1a(human opt)}- |
| | 2xC/Dbox-2xU1a_RBS/25 ng |
| (5) ELN-CD63-U + | (1) p25 pRP[Exp]-Bsd-EF1A > {CD63-L7ae} (RNA packer)/150 ng |
| PTGFRN (puro) + | (2) p27 pLV[Exp]-Puro-CMV + intron > {PTGFRN-U1a}(RNA packer)/ |
| (bsd) | 150 ng |
| | (3) p30 pVB230615-1543puy_{—}Myo_{—}Cnx43_{—}Neomycin/150 ng |
| | (4) p16 pLV[Exp]-Hygro-EF1A > {ELN1-CD-U1a(human opt)}- |
| | 2xC/Dbox-2xU1a_RBS/150 ng |
| (6) ELN-CD63-L + | (1) p25 pRP[Exp]-Bsd-EF1A > {CD63-L7ae} (RNA packer)/ 150 ng |
| CD81 (puro) + | (2) p28 pLV[Exp]-Puro-CMV + intron > {CD81-U1a}/150 ng |
| (bsd) | (3) p30 pVB230615-1543puy_{—}Myo_{—}Cnx43_{—}Neomycin/150 ng |
| | (4) p16 pLV[Exp]-Hygro-EF1A > {ELN1-CD-U1a(human opt)}- |
| | 2xC/Dbox-2xU1a_RBS/150 ng |

Note:
underline denotes the RNA packaging plasmid and italics the enhancer plasmid. mRNA expressing construct shown in black font.

TABLE 3
Triple/quadruplicate drug combinations for selection
of stable ELN mRNA-EV producing cells.

| Concentration | Drug concentration |
|---|---|
| (1) Conc-1 | (1) Puromycin (1 μg/ml) |
| | (2) Blastocydin (0.2 μg/ml) |
| | (3) G418 (200 μg/ml) |
| | (4) Hygromycin (50 μg/ml) |
| (2) Conc-2 | (1) Puromycin (10 μg/ml) |
| | (2) Blastocydin (1.0 μg/ml) |
| | (3) G410 (300 μg/ml) |
| | (4) Hygromycin (575 μg/ml) |
| (3) Conc-3 | (1) Puromycin (15 g/ml) |
| | (2) Blastocydin (2.5 μg/ml) |
| | (3) G410 (400 μg/ml) |
| | (4) Hygromycin (100 μg/ml) |
| (4) Conc-4 | (1) Puromycin (20 μg/ml) |
| | (2) Blastocydin (5.0 μg/ml) |
| | (3) G410 (500 μg/ml) |
| | (4) Hygromycin (150 μg/ml) |

TABLE 4
Primers used for screening mRNA expression for (ELN) expression by qRTPCR.
Standardize using Beta actin qRTPCR.

| Primer | | |
|---|---|---|
| and | | |
| mRNA | | |
| amplified | Forward primer (5′-3′) | Reverse primer (5′-3′) |
| (1) CD- | >CD-UR_F1 | >CD-UR R1 |
| UR | GTGATCCGAAAGGTGAGTACC | AGGATAATCCGGAGTGCAATG |
| domain | | |
| (universal | | |
| primers | | |
| for all | | |
| RNAs) | | |
| (2) ELN | ELN_F1 | >>ELN_R1 |
| | GGGAAGTTGCCCTATGGTTATG | GCAGCACCTGCTCCAAAT |
| (3) Beta | >Beta Actin F | >Beta Actin R |
| actin | ACAGCCTGGATAGCAACG | CACCAACTGGGACGACAT |
| mRNA | | |

### Results

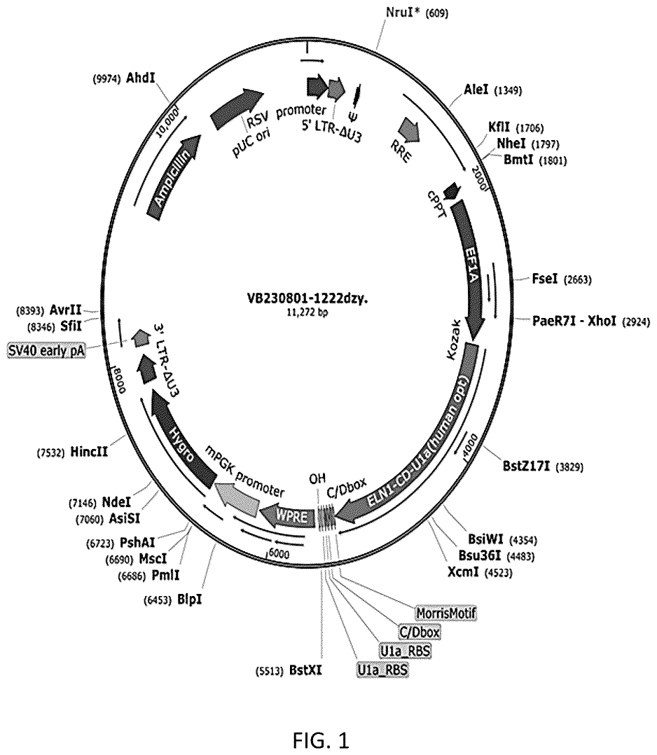

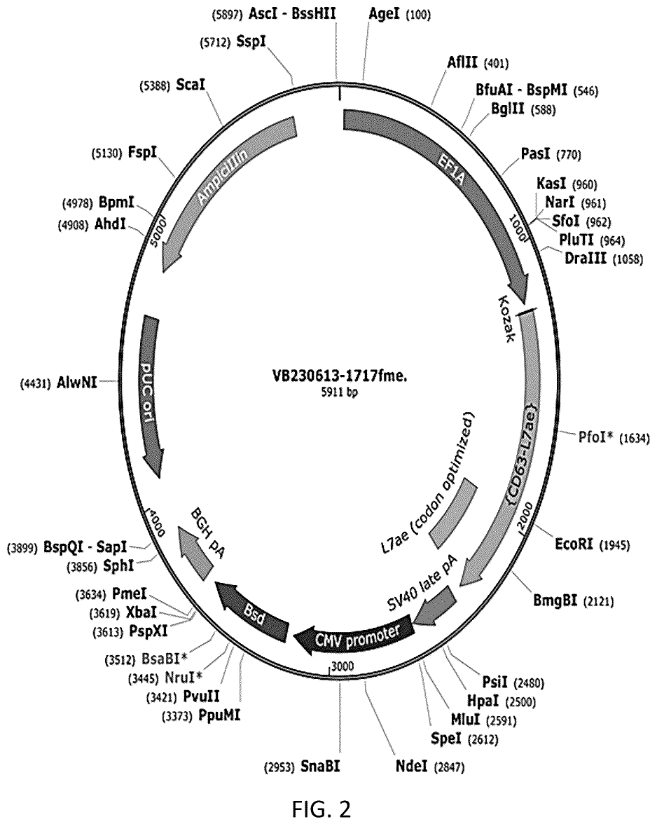

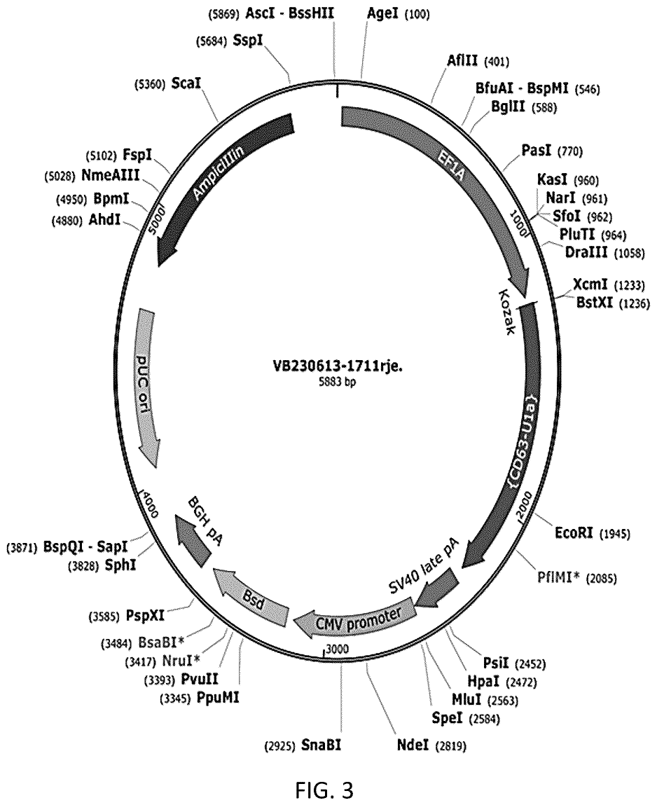

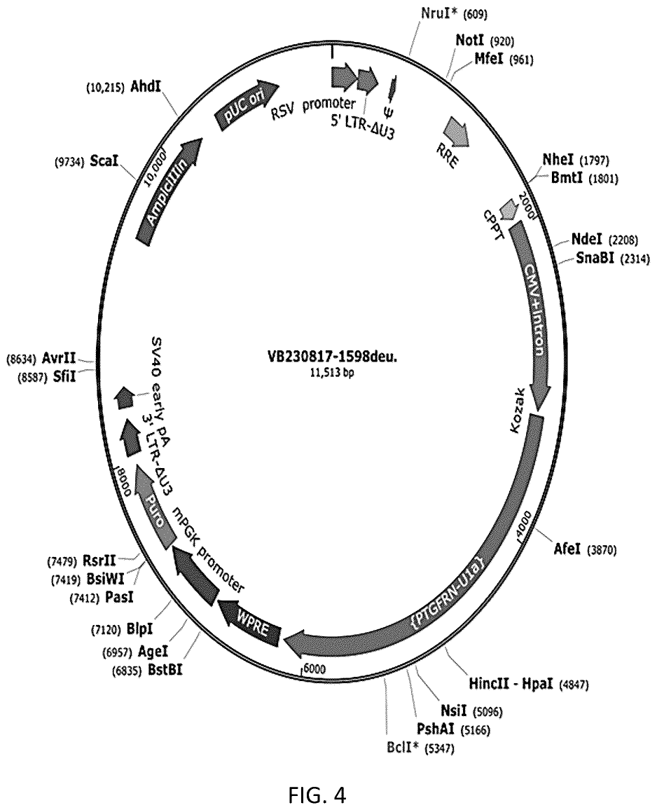

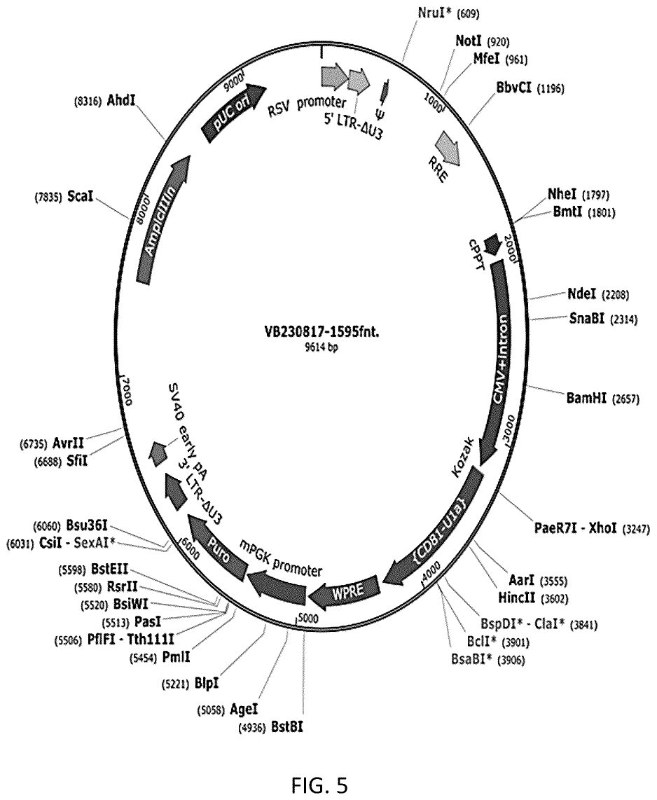

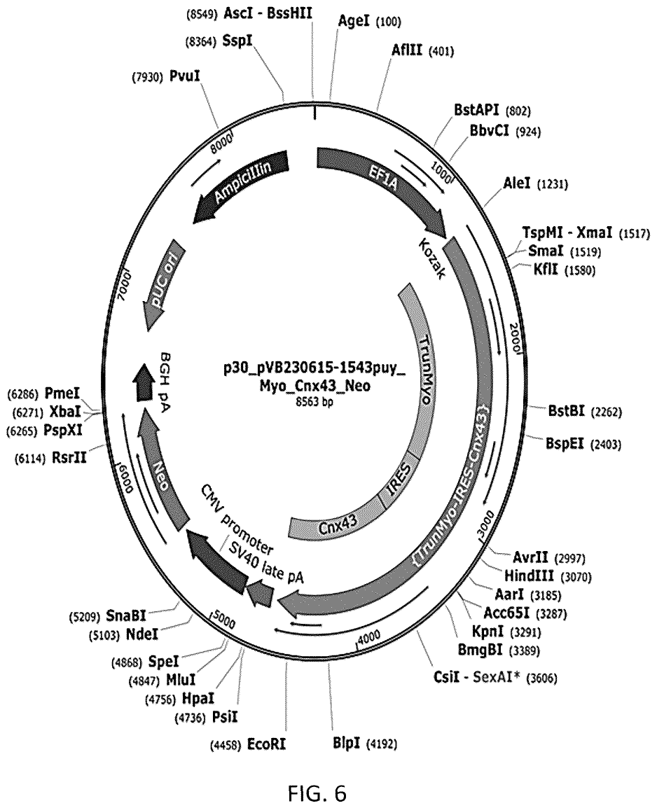

Several stable ELN exosome producing HEK293 cell lines were generated to express ELN (Table 5). The ELN expressing plasmid (FIG. 1), RNA packaging L7ae based (FIG. 2) and U1a (FIGS. 3-5) and Exosome enhancing plasmids (FIG. 6) were co-transfected into producer cells. The resultant cells were co-cultured with recipient cells and uptake of ELN determined by qRT-PCR 72-96 hrs later.

TABLE 5
Elastin coding sequence 5′→3′

atggcgggcctgaccgcggcggcgccgcgcccgggcgtgctgctgctgctgctgagcattctgcatccgagccgcccgggcggc
gtgccgggcgcgattccgggcggcgtgccgggcggcgtgttttatccgggcgcgggcctgggcgcgctgggcggcggcgcgctg
ggcccgggcggcaaaccgctgaaaccggtgccgggcggcctggcgggcgcgggcctgggcgcgggcctgggcgcgtttccgg
cggtgacctttccgggcgcgctggtgccgggcggcgtggcggatgcggcggcggcgtataaagcggcgaaagcgggcgcgggc
ctgggcggcgtgccgggcgtgggcggcctgggcgtgagcgcgggcgcggtggtgccgcagccgggcgcgggcgtgaaaccgg
gcaaagtgccgggcgtgggcctgccgggcgtgtatccgggcggcgtgctgccgggcgcgcgctttccgggcgtgggcgtgctgc
cgggcgtgccgaccggcgcgggcgtgaaaccgaaagcgccgggcgtgggcggcgcgtttgcgggcattccgggcgtgggcccg
tttggcggcccgcagccgggcgtgccgctgggctatccgattaaagcgccgaaactgccgggcggctatggcctgccgtataccac
cggcaaactgccgtatggctatggcccgggcggcgtggcgggcgcggcgggcaaagcgggctatccgaccggcaccggcgtgg
gcccgcaggcggcggcggcggcggcggcgaaagcggcggcgaaatttggcgcgggcgcggcgggcgtgctgccgggcgtgg
gcggcgcgggcgtgccgggcgtgccgggcgcgattccgggcattggcggcattgcgggcgtgggcaccccggcggcggcggc
ggcggcggcggcggcggcgaaagcggcgaaatatggcgcggcggcgggcctggtgccgggcggcccgggctttggcccggg
cgtgggggcgtgccgggcgcgggcgtgccgggcgtgggcgtgccgggcgcgggcattccggtggtgccgggcgcgggcattc
cgggcgcggcggtgccgggcgtggtgagcccggaagcggcggcgaaagcggcggcgaaagcggcgaaatatggcgcgcgcc
cgggcgtgggcgtgggcggcattccgacctatggcgtgggcgcgggcggctttccgggctttggcgtgggcgtgggcggcattcc
gggcgtggcgggcgtgccgggcgtgggcggcgtgccgggcgtgggcggcgtgccgggcgtgggcattagcccggaagcgcag
gcggcggcggcggcgaaagcggcgaaatatggcgtgggcaccccggcggcggcggcggcgaaagcggcggcgaaagcggc
gcagtttggcctggtgccgggcgtgggcgtggcgccgggcgtgggcgtggcgccgggcgtgggcgtggcgccgggcgtgggcc
tggcgccgggcgtgggcgtggcgccgggcgtgggcgtggcgccgggcgtgggcgtggcgccgggcattggcccgggcggcgt
ggcggcggcggcgaaaagcgcggcgaaagtggcggcgaaagcgcagctgcgcgcggcggcgggcctgggcgcgggcattcc
gggcctgggcgtgggcgtgggcgtgccgggcctgggcgtgggcgcgggcgtgccgggcctgggcgtgggcgcgggcgtgccg
ggctttggcgcggtgccgggcgcgctggcggcggcgaaagcggcgaaatatggcgcggcggtgccgggcgtgctgggggcct
gggcgcgctgggcggcgtgggcattccgggcggcgtggtgggcgcgggcccggcggcggcggcggcggcggcgaaagcggc
ggcgaaagcggcgcagtttggcctggtgggcgcggcgggcctgggcggcctgggcgtgggcggcctgggcgtgccgggcgtgg
gcggcctgggcggcattccgccggcggcggcggcgaaagcggcgaaatatggcgcggcgggcctgggcggcgtgctgggcgg
cgcgggccagtttccgctgggcggcgtggcggcgcgcccgggctttggcctgagcccgatttttccgggcggcgcgtgcctgggca
aagcgtgcggccgcaaacgcaaaGCGCAGCGCGCGCAGCGCatagactttgggtaccgtgatccgaaaggtgagt
acccGTGAGATCCgggtaccgtgatccgaaaggtgagtacccTTCGAATCCATTGCACTCCGGATTT
TTGAATTGGAATCCATTGCACTCCGGATTATCCTTCGCTGCAGATATCCAGCACA
GTGGCatagGCGCAGCGCGCGCAGCGCactttgggtaccgtgatccgaaaggtgagtacccGGATCCggg
taccgtgatccgaaaggtgagtacccTTCGAATCCATTGCACTCCGGATTTTTGAATTGGAATCC
ATTGCACTCCGGATTATCCTTCGCTGCAGATATCCAGCACAGTGGC

### Example: EV Transfer of mRNA

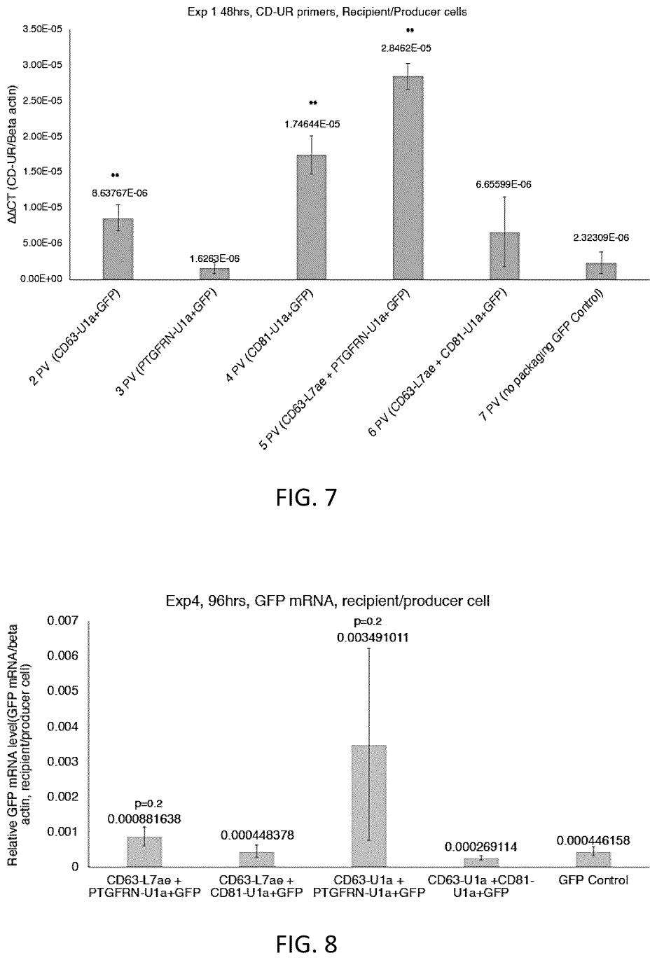

Various combinations of packaging constructs, e.g. CD63-U1a, PTGFRN-U1a, CD81-U1a, CD63-L7ae, or a combination thereof were assessed for delivery of a GFP mRNA. All fusion constructs were found to be functional, and the ability to package GFP mRNAs (FIG. 7 and FIG. 8), suggesting that CD63, CD81 and PTGREN U1a fusions are functional standouts in the RNex platform that can be used to package therapeutic RNAs, antisense non-coding RNAs and mRNAs or various combinations or RNAs. Collectively, these data suggest that our RNex packaging system is functional relative to the control L7ae system, but superior as the U1a/UR system uses endogenous Human protein components (e.g. U1a), whereas the L7ae is a foreign RNA binding protein.

It can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

1. A pharmaceutical composition comprising an exosome encapsulating one or more cargo RNAs and one or more fusion proteins,
wherein the fusion protein comprises an exosome-associated transmembrane protein fused to a packaging protein;
wherein the cargo RNA comprises a package RNA and a packaging domain;
wherein the packaging protein is capable of binding to the packaging domain to form a fusion protein cargo RNA complex; and
wherein the package RNA comprises an elastin protein encoding polynucleotide
wherein nucleotide sequence of the elastin protein encoding polynucleotide is at least about 80%, about 85%, about 90%, about 95% or about 100% identical to the nucleotide sequence of an elastin gene or an elastin messenger RNA (mRNA).

2. The pharmaceutical composition of claim 1, wherein the packaging protein is endogenous to a subject.

3. The pharmaceutical composition of claim 1, wherein the packaging protein comprises U1a protein.

4. The pharmaceutical composition of claim 1, wherein nucleotide sequence of the elastin protein encoding polynucleotide is at least about 80%, about 85%, about 90%, about 95% or about 100% identical to SEQ ID NO. 2 or SEQ ID NO. 3.

5. The pharmaceutical composition of claim 1, wherein the elastin gene or the elastin mRNA comprises any naturally occurring isoform thereof.

6. The pharmaceutical composition of claim 5, wherein the elastin mRNA isoforms are derived from alternative splicing of the elastin gene.

7. The pharmaceutical composition of claim 1, wherein the packaging domain comprises an UR domain, wherein the nucleotide sequence of the UR domain is at least about 80%, about 85%, about 90%, about 95% or about 100% identical to SEQ ID NO. 10 or SEQ ID NO. 11.

8. The pharmaceutical composition of claim 1, wherein the one or more cargo RNAs further comprises one or more OH domain, MorrisMotif domain, or a combination thereof, wherein the nucleotide sequence of OH domain is at least about 80%, about 85%, about 90%, about 95% or about 100% identical to SEQ ID NO. 12, and wherein the nucleotide sequence of MorrisMotif domain is at least about 80%, about 85%, about 90%, about 95% or about 100% identical to SEQ ID NO. 13.

9. The pharmaceutical composition of claim 8, wherein the nucleotide sequence of at least one of the one or more cargo RNAs is at least about 80%, about 85%, about 90%, about 95% or about 100% identical to SEQ ID NO. 15.

10. The pharmaceutical composition of claim 1, wherein the fusion protein comprises CD63-U1a, CD81-U1a, PTGFRN-U1a, or a combination thereof.

11. The pharmaceutical composition of claim 1, wherein the amino acid sequence of the fusion protein is at least about 80%, about 85%, about 90%, about 95% or about 100% identical or similar to SEQ ID NO. 5, SEQ ID NO. 7, SEQ ID NO. 9, or a combination thereof.

12. The pharmaceutical composition of claim 1, further comprises a modified myoferlin protein.

13. The pharmaceutical composition of claim 12, wherein the modified myoferlin protein comprises C2F, C2G, transmembrane domain of the myoferlin protein, or a combination thereof.

14. The pharmaceutical composition of claim 13, wherein amino acid sequence of the modified myoferlin protein is at least about 80%, about 85%, about 90%, about 95% or about 100% identical or similar to SEQ ID NO. 28.

15. The pharmaceutical composition of claim 12, wherein the modified myoferlin protein comprises C2A, FerA, FerB, DysFN, transmembrane domain of the myoferlin protein, or a combination thereof.

16. The pharmaceutical composition of claim 12, wherein the modified myoferlin protein further comprises a connexin 43 protein.

17. The pharmaceutical composition of claim 16, wherein the connexin 43 protein comprises a S368A mutation.

18. The pharmaceutical composition of claim 16, wherein amino acid sequence of the connexin 43 protein is at least about 80%, about 85%, about 90%, about 95% or about 100% identical or similar to SEQ ID NO. 41.

19. The pharmaceutical composition of claim 1, wherein the exosome is prepared from an exosome-based packaging and delivery system comprising an exosome producing cell, one or more cargo RNA encoding plasmids that encodes the package RNA and the packaging domain, and one or more fusion protein encoding plasmids that encodes the exosome-associated transmembrane protein fused to the packaging protein.

20. The pharmaceutical composition of claim 19, wherein the exosome-based packaging and delivery system further comprises a plasmid comprising a modified myoferlin protein.

21. A method of treatment of elastin-associated disease of a subject comprising the step of administration of a therapeutically effective amount of the pharmaceutical composition of claim 1 to the subject.

22. The method of claim 21, wherein the elastin gene expression in the subject comprises disease-associated deletions or mutations.

23. The method of claim 21, wherein the elastin-associated disease comprises supravalvular aortic stenosis (SVAS), autosomal dominant cutis laxa, Marfan syndrome, alpha1-antitrypsin deficiency-related emphysema, atherosclerosis, Buschke-Ollendorff syndrome, Menkes syndrome, pseudoxanthoma elasticum, and Williams syndrome.

24. The method of claim 21, wherein the subject is deficient in elastin.

25. The method of claim 21, wherein the elastin-associated disease comprises scaring, burned, saggy or wrinkly skin.

26. The method of claim 21, wherein the step of administration of a therapeutically effective amount of the pharmaceutical composition 1 to the subject comprises topical, oral, or parenteral forms of administration.

27. The method of claim 21, wherein the administration of the therapeutically effective amount of the pharmaceutical composition to the subject comprises microneedle administration.


SEQUENCE LISTING (42 sequences)

SEQ ID NO: 1 - protein, 724 aa, synthetic construct
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 2 - DNA, 2174 bp, synthetic construct
  source 1..2174: mol_type = other DNA
atggcgggcctgaccgcggcggcgccgcgcccgggcgtgctgctgctgctgctgagcattctgcatccgagccgcccgggcggcgtgccgggcgcgattccgggcggcgtgccgggcggcgtgttttatccgggcgcgggcctgggcgcgctgggcggcggcgcgctgggcccgggcggcaaaccgctgaaaccggtgccgggcggcctggcgggcgcgggcctgggcgcgggcctgggcgcgtttccggcggtgacctttccgggcgcgctggtgccgggcggcgtggcggatgcggcggcggcgtataaagcggcgaaagcgggcgcgggcctgggcggcgtgccgggcgtgggcggcctgggcgtgagcgcgggcgcggtggtgccgcagccgggcgcgggcgtgaaaccgggcaaagtgccgggcgtgggcctgccgggcgtgtatccgggcggcgtgctgccgggcgcgcgctttccgggcgtgggcgtgctgccgggcgtgccgaccggcgcgggcgtgaaaccgaaagcgccgggcgtgggcggcgcgtttgcgggcattccgggcgtgggcccgtttggcggcccgcagccgggcgtgccgctgggctatccgattaaagcgccgaaactgccgggcggctatggcctgccgtataccaccggcaaactgccgtatggctatggcccgggcggcgtggcgggcgcggcgggcaaagcgggctatccgaccggcaccggcgtgggcccgcaggcggcggcggcggcggcggcgaaagcggcggcgaaatttggcgcgggcgcggcgggcgtgctgccgggcgtgggcggcgcgggcgtgccgggcgtgccgggcgcgattccgggcattggcggcattgcgggcgtgggcaccccggcggcggcggcggcggcggcggcggcggcgaaagcggcgaaatatggcgcggcggcgggcctggtgccgggcggcccgggctttggcccgggcgtggtgggcgtgccgggcgcgggcgtgccgggcgtgggcgtgccgggcgcgggcattccggtggtgccgggcgcgggcattccgggcgcggcggtgccgggcgtggtgagcccggaagcggcggcgaaagcggcggcgaaagcggcgaaatatggcgcgcgcccgggcgtgggcgtgggcggcattccgacctatggcgtgggcgcgggcggctttccgggctttggcgtgggcgtgggcggcattccgggcgtggcgggcgtgccgggcgtgggcggcgtgccgggcgtgggcggcgtgccgggcgtgggcattagcccggaagcgcaggcggcggcggcggcgaaagcggcgaaatatggcgtgggcaccccggcggcggcggcggcgaaagcggcggcgaaagcggcgcagtttggcctggtgccgggcgtgggcgtggcgccgggcgtgggcgtggcgccgggcgtgggcgtggcgccgggcgtgggcctggcgccgggcgtgggcgtggcgccgggcgtgggcgtggcgccgggcgtgggcgtggcgccgggcattggcccgggcggcgtggcggcggcggcgaaaagcgcggcgaaagtggcggcgaaagcgcagctgcgcgcggcggcgggcctgggcgcgggcattccgggcctgggcgtgggcgtgggcgtgccgggcctgggcgtgggcgcgggcgtgccgggcctgggcgtgggcgcgggcgtgccgggctttggcgcggtgccgggcgcgctggcggcggcgaaagcggcgaaatatggcgcggcggtgccgggcgtgctgggcggcctgggcgcgctgggcggcgtgggcattccgggcggcgtggtgggcgcgggcccggcggcggcggcggcggcggcgaaagcggcggcgaaagcggcgcagtttggcctggtgggcgcggcgggcctgggcggcctgggcgtgggcggcctgggcgtgccgggcgtgggcggcctgggcggcattccgccggcggcggcggcgaaagcggcgaaatatggcgcggcgggcctgggcggcgtgctgggcggcgcgggccagtttccgctgggcggcgtggcggcgcgcccgggctttggcctgagcccgatttttccgggcggcgcgtgcctgggcaaagcgtgcggccgcaaacgcaaagc

SEQ ID NO: 3 - DNA, 3395 bp, synthetic construct
  source 1..3395: mol_type = other DNA
gcatttctccccgagatggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctggtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatgagcttcctaggacccctgactcacgacctcatcaacgttggtgctactgcttggtggagaatgtaaaccctttgtaaccccatcccatgcccctccgactccccaccccaggagggaacgggcaggccgggcggccttgcagatccacagggcaaggaaacaagaggggagcggccaagtgccccgaccaggaggccccctacttcagaggcaagggccatgtggtcctggccccccaccccatcccttcccacctaggagctccccctcacacagcctccatctccaggggaacttggtgctacacgctggtgctcttatcttcctggggggagggaggagggaagggtggcccctcggggaaccccctacctggggctcctctaaagatggtgcagacacttcctgggcagtcccagctccccctgcccaccaggacccaccgttggctgccatccagttggtacccaagcacctgaagcctcaaagctggattcgctctagcatccctcctctcctgggtccacttggccgtctcctccccaccgatcgctgttccccacatctggggcgcttttgggttggaaaaccaccccacactgggaatagccaccttgcccttgtagaatccatccgcccatccgtccattcatccatcggtccgtccatccatgtccccagttgaccgcccggcaccactagctggctgggtgcacccaccatcaacctggttgacctgtcatggccgcctgtgccctgcctccacccccatcctacactcccccagggcgtgcggggctgtgcagactgggtgccaggcatctcctccccacccggggtgtccccacatgcagtactgtataccccccatccctccctcggtccactgaacttcagagcagttcccattcctgccccgcccatctttttgtgtctcgctgtgatagatcaataaatattttattttttgtcctggatatttggggattatttttgattgttgatattctcttttggttttattgttgtggttcattgaaaaaaaaagataatttttttttctgatccggggagctgtatccccagtagaaaaaacattttaatcactctaatataactctggatgaaacacacctttttttttaataagaaaagagaattaactgcttcagaaatgactaataaatgaaaaacctttaaaggaaa

SEQ ID NO: 4 - DNA, 1029 bp, synthetic construct
  source 1..1029: mol_type = other DNA
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatggaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaa

SEQ ID NO: 5 - protein, 343 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVMEFGGGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMK

SEQ ID NO: 6 - DNA, 1056 bp, synthetic construct
  source 1..1056: mol_type = other DNA
atgtccggactcagatctcgagctcaagcttccggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtacgaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaataa

SEQ ID NO: 7 - protein, 351 aa, synthetic construct
MSGLRSRAQASGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYEFGGGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMK

SEQ ID NO: 8 - DNA, 2955 bp, synthetic construct
  source 1..2955: mol_type = other DNA
atggggcgcctggcctccaggccgctgctgctggcgctcctgtcgttggctctttgccgagggcgtgtggtgagagtccccacagcgaccctggttcgagtggtgggcactgagctggtcatcccctgcaacgtcagtgactatgatggccccagcgagcaaaactttgactggagcttctcatctttggggagcagctttgtggagcttgcaagcacctgggaggtggggttcccagcccagctgtaccaggagcggctgcagaggggcgagatcctgttaaggcggactgccaacgacgccgtggagctccacataaagaacgtccagccttcagaccaaggccactacaaatgttcaacccccagcacagatgccactgtccagggaaactatgaggacacagtgcaggttaaagtgctggccgactccctgcacgtgggccccagcgcgcggcccccgccgagcctgagcctgcgggagggggagcccttcgagctgcgctgcaccgccgcctccgcctcgccgctgcacacgcacctggcgctgctgtgggaggtgcaccgcggcccggccaggcggagcgtcctcgccctgacccacgagggcaggttccacccgggcctggggtacgagcagcgctaccacagtggggacgtgcgcctcgacaccgtgggcagcgacgcctaccgcctctcagtgtcccgggctctgtctgccgaccagggctcctacaggtgtatcgtcagcgagtggatcgccgagcagggcaactggcaggaaatccaagaaaaggccgtggaagttgccaccgtggtgatccagccatcagttctgcgagcagctgtgcccaagaatgtgtctgtggctgaaggaaaggaactggacctgacctgtaacatcacaacagaccgagccgatgacgtccggcccgaggtgacgtggtccttcagcaggatgcctgacagcaccctacctggctcccgcgtgttggcgcggcttgaccgtgattccctggtgcacagctcgcctcatgttgctttgagtcatgtggatgcacgctcctaccatttactggttcgggatgttagcaaagaaaactctggctactattactgccacgtgtccctgtgggcacccggacacaacaggagctggcacaaagtggcagaggccgtgtcttccccagctggtgtgggtgtgacctggctagaaccagactaccaggtgtacctgaatgcttccaaggtccccgggtttgcggatgaccccacagagctggcatgccgggtggtggacacgaagagtggggaggcgaatgtccgattcacggtttcgtggtactacaggatgaaccggcgcagcgacaatgtggtgaccagcgagctgcttgcagtcatggacggggactggacgctaaaatatggagagaggagcaagcagcgggcccaggatggagactttattttttctaaggaacatacagacacgttcaatttccggatccaaaggactacagaggaagacagaggcaattattactgtgttgtgtctgcctggaccaaacagcggaacaacagctgggtgaaaagcaaggatgtcttctccaagcctgttaacatattttgggcattagaagattccgtgcttgtggtgaaggcgaggcagccaaagcctttctttgctgccggaaatacatttgagatgacttgcaaagtatcttccaagaatattaagtcgccacgctactctgttctcatcatggctgagaagcctgtcggcgacctctccagtcccaatgaaacgaagtacatcatctctctggaccaggattctgtggtgaagctggagaattggacagatgcatcacgggtggatggcgttgttttagaaaaagtgcaggaggatgagttccgctatcgaatgtaccagactcaggtctcagacgcagggctgtaccgctgcatggtgacagcctggtctcctgtcaggggcagcctttggcgagaagcagcaaccagtctctccaatcctattgagatagacttccaaacctcaggtcctatatttaatgcttctgtgcattcagacacaccatcagtaattcggggagatctgatcaaattgttctgtatcatcactgtcgagggagcagcactggatccagatgacatggcctttgatgtgtcctggtttgcggtgcactcttttggcctggacaaggctcctgtgctcctgtcttccctggatcggaagggcatcgtgaccacctcccggagggactggaagagcgacctcagcctggagcgcgtgagtgtgctggaattcttgctgcaagtgcatggctccgaggaccaggactttggcaactactactgttccgtgactccatgggtgaagtcaccaacaggttcctggcagaaggaggcagagatccactccaagcccgtttttataactgtgaagatggatgtgctgaacgccttcaagtatcccttgctgatcggcgtcggtctgtccacggtcatcgggctcctgtcctgtctcatcgggtactgcagctcccactggtgttgtaagaaggaggttcaggagacacggcgcgagcgccgcaggctcatgtcgatggagatggacgaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaatag

SEQ ID NO: 9 - protein, 984 aa, synthetic construct
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMDEFGGGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMK

SEQ ID NO: 10 - DNA, 20 bp, synthetic construct
  source 1..20: mol_type = other DNA
aatccattgcactccggatt

SEQ ID NO: 11 - DNA, 21 bp, synthetic construct
  source 1..21: mol_type = other DNA
aatccattgcactccggattt

SEQ ID NO: 12 - DNA, 23 bp, synthetic construct
  source 1..23: mol_type = other DNA
ctgcagatatccagcacagtggc

SEQ ID NO: 13 - DNA, 18 bp, synthetic construct
  source 1..18: mol_type = other DNA
gcgcagcgcgcgcagcgc

SEQ ID NO: 14 - DNA, 42 bp, synthetic construct
  source 1..42: mol_type = other DNA
cgcctcccgggttcaagcgattctcctgcctcagcctcccga

SEQ ID NO: 15 - DNA, 2527 bp, synthetic construct
  source 1..2527: mol_type = other DNA
atggcgggcctgaccgcggcggcgccgcgcccgggcgtgctgctgctgctgctgagcattctgcatccgagccgcccgggcggcgtgccgggcgcgattccgggcggcgtgccgggcggcgtgttttatccgggcgcgggcctgggcgcgctgggcggcggcgcgctgggcccgggcggcaaaccgctgaaaccggtgccgggcggcctggcgggcgcgggcctgggcgcgggcctgggcgcgtttccggcggtgacctttccgggcgcgctggtgccgggcggcgtggcggatgcggcggcggcgtataaagcggcgaaagcgggcgcgggcctgggcggcgtgccgggcgtgggcggcctgggcgtgagcgcgggcgcggtggtgccgcagccgggcgcgggcgtgaaaccgggcaaagtgccgggcgtgggcctgccgggcgtgtatccgggcggcgtgctgccgggcgcgcgctttccgggcgtgggcgtgctgccgggcgtgccgaccggcgcgggcgtgaaaccgaaagcgccgggcgtgggcggcgcgtttgcgggcattccgggcgtgggcccgtttggcggcccgcagccgggcgtgccgctgggctatccgattaaagcgccgaaactgccgggcggctatggcctgccgtataccaccggcaaactgccgtatggctatggcccgggcggcgtggcgggcgcggcgggcaaagcgggctatccgaccggcaccggcgtgggcccgcaggcggcggcggcggcggcggcgaaagcggcggcgaaatttggcgcgggcgcggcgggcgtgctgccgggcgtgggcggcgcgggcgtgccgggcgtgccgggcgcgattccgggcattggcggcattgcgggcgtgggcaccccggcggcggcggcggcggcggcggcggcggcgaaagcggcgaaatatggcgcggcggcgggcctggtgccgggcggcccgggctttggcccgggcgtggtgggcgtgccgggcgcgggcgtgccgggcgtgggcgtgccgggcgcgggcattccggtggtgccgggcgcgggcattccgggcgcggcggtgccgggcgtggtgagcccggaagcggcggcgaaagcggcggcgaaagcggcgaaatatggcgcgcgcccgggcgtgggcgtgggcggcattccgacctatggcgtgggcgcgggcggctttccgggctttggcgtgggcgtgggcggcattccgggcgtggcgggcgtgccgggcgtgggcggcgtgccgggcgtgggcggcgtgccgggcgtgggcattagcccggaagcgcaggcggcggcggcggcgaaagcggcgaaatatggcgtgggcaccccggcggcggcggcggcgaaagcggcggcgaaagcggcgcagtttggcctggtgccgggcgtgggcgtggcgccgggcgtgggcgtggcgccgggcgtgggcgtggcgccgggcgtgggcctggcgccgggcgtgggcgtggcgccgggcgtgggcgtggcgccgggcgtgggcgtggcgccgggcattggcccgggcggcgtggcggcggcggcgaaaagcgcggcgaaagtggcggcgaaagcgcagctgcgcgcggcggcgggcctgggcgcgggcattccgggcctgggcgtgggcgtgggcgtgccgggcctgggcgtgggcgcgggcgtgccgggcctgggcgtgggcgcgggcgtgccgggctttggcgcggtgccgggcgcgctggcggcggcgaaagcggcgaaatatggcgcggcggtgccgggcgtgctgggcggcctgggcgcgctgggcggcgtgggcattccgggcggcgtggtgggcgcgggcccggcggcggcggcggcggcggcgaaagcggcggcgaaagcggcgcagtttggcctggtgggcgcggcgggcctgggcggcctgggcgtgggcggcctgggcgtgccgggcgtgggcggcctgggcggcattccgccggcggcggcggcgaaagcggcgaaatatggcgcggcgggcctgggcggcgtgctgggcggcgcgggccagtttccgctgggcggcgtggcggcgcgcccgggctttggcctgagcccgatttttccgggcggcgcgtgcctgggcaaagcgtgcggccgcaaacgcaaagcgcagcgcgcgcagcgcatagactttgggtaccgtgatccgaaaggtgagtacccgtgagatccgggtaccgtgatccgaaaggtgagtacccttcgaatccattgcactccggatttttgaattggaatccattgcactccggattatccttcgctgcagatatccagcacagtggcataggcgcagcgcgcgcagcgcactttgggtaccgtgatccgaaaggtgagtacccggatccgggtaccgtgatccgaaaggtgagtacccttcgaatccattgcactccggatttttgaattggaatccattgcactccggattatccttcgctgcagatatccagcacagtggc

SEQ ID NO: 16 - protein, 124 aa, synthetic construct
QFRELPDSVPQECTVRIYIVRGLELQPQDNNGLCDPYIKITLGKKVIEDRDHYIPNTLNPVFGRMYELSCYLPQEKDLKISVYDYDTFTRDEKVGETIIDLENRFLSRFGSHCGIPEEYCVSGV

SEQ ID NO: 17 - protein, 132 aa, synthetic construct
PFNITPRKAKKYYLRVIIWNTKDVILDEKSITGEEMSDIYVKGWVPGNEENKQKTDVHYRSLDGEGNFNWRFVFPFDYLPAEQLCIVAKKEHFWSIDQTEFRIPPRLIIQIWDNDKFSLDDYLGFLELDLRH

SEQ ID NO: 18 - protein, 119 aa, synthetic construct
PDLKAMNPLKAKTASLFEQKSMKGWWPCYAEKDGARVMAGKVEMTLEILNEKEADERPAGKGRDEPNMNPKLDLPNRPETSFLWFTNPCKTMKFIVWRRFKWVIIGLLFLLILLLFVAV

SEQ ID NO: 19 - protein, 136 aa, synthetic construct
MLRVIVESASNIPKTKFGKPDPIVSVIFKDEKKKTKKVDNELNPVWNEILEFDLRGIPLDFSSSLGIIVKDFETIGQNKLIGTATVALKDLTGDQSRSLPYKLISLLNERGQDTGATIDLVIGYDPPSAPHPNDLS

SEQ ID NO: 20 - protein, 58 aa, synthetic construct
LQTNIEALKSGIQGKIPANQLAELWLKLIDEVIEDTRYTLPLTEGKANVTVLDTQIRK

SEQ ID NO: 21 - protein, 72 aa, synthetic construct
WLDKLMQLTEEPQNSMPDIIIWMIRGEKRLAYARIPAHQVLYSTSGENASGKYCGKTQTIFLKYPQEKNNGP

SEQ ID NO: 22 - protein, 58 aa, synthetic construct
AVEKKFNSFAEGTFTVFAEMYENQALMFGKWGTSGLVGRHKFSDVTGKIKLKREFFLP

SEQ ID NO: 23 - protein, 57 aa, synthetic construct
DPERSLLTEADAGHTEFTDEVYQNESRYPGGDWKPAEDTYTDANGDKAASPSELTCP

SEQ ID NO: 24 - protein, 110 aa, synthetic construct
PQDFQIRVRVIEGRQLSGNNIRPVVKVHVCGQTHRTRIKRGNNPFFDELFFYNVNMTPSELMDEIISIRVYNSHSLRADCLMGEFKIDVGFVYDEPGHAVMRKWLLLNDP

SEQ ID NO: 25 - protein, 164 aa, synthetic construct
TFLLKIYRAEDIPQMDDAFSQTVKEIFGGNADKKNLVDPFVEVSFAGKKVCTNIIEKNANPEWNQVVNLQIKFPSVCEKIKLTIYDWDRLTKNDVVGTTYLHLSKIAASGGEVEDFSSSGTGAASYTVNTGETEVGFVPTFGPCYLNLYGSPREYTGFPDPYDE

SEQ ID NO: 26 - protein, 134 aa, synthetic construct
TPIVSCNFDRVYIYHLRCYVYQARNLLALDKDSFSDPYAHICFLHRSKTTEIIHSTLNPTWDQTIIFDEVEIYGEPQTVLQNPPKVIMELFDNDQVGKDEFLGRSIFSPVVKLNSEMDITPKLLWHPVMNGDKA

SEQ ID NO: 27 - protein, 77 aa, synthetic construct
RNMKNFQMASITSPSLVVECGGERVESVVIKNLKKTPNFPSSVLFMKVFLPKEELYMPPLVIKVIDHRQFGRKPVVG

SEQ ID NO: 28 - protein, 537 aa, synthetic construct
MVPAPPRQFRELPDSVPQECTVRIYIVRGLELQPQDNNGLCDPYIKITLGKKVIEDRDHYIPNTLNPVFGRMYELSCYLPQEKDLKISVYDYDTFTRDEKVGETIIDLENRFLSRFGSHCGIPEEYCVSGVNTWRDQLRPTQLLQNVARFKGFPQPILSEDGSRIRYGGRDYSLDEFEANKILHQHLGAPEERLALHILRTQGLVPEHVETRTLHSTFQPNISQGKLQMWVDVFPKSLGPPGPPFNITPRKAKKYYLRVIIWNTKDVILDEKSITGEEMSDIYVKGWVPGNEENKQKTDVHYRSLDGEGNFNWRFVFPFDYLPAEQLCIVAKKEHFWSIDQTEFRIPPRLIIQIWDNDKFSLDDYLGFLELDLRHTIIPAKSPEKCRLDMIPDLKAMNPLKAKTASLFEQKSMKGWWPCYAEKDGARVMAGKVEMTLEILNEKEADERPAGKGRDEPNMNPKLDLPNRPETSFLWFTNPCKTMKFIVWRRFKWVIIGLLFLLILLLFVAVLLYSLPNYLSMKIVKPNVYPYDVPDYA

SEQ ID NO: 29 - DNA, 408 bp, synthetic construct
  source 1..408: mol_type = other DNA
atgctgcgagtgattgtggaatctgccagcaatatccctaaaacgaaatttggcaagccggatcctattgtttctgtcatttttaaggatgagaaaaagaaaacaaagaaagttgataatgaattgaaccctgtctggaatgagattttggagtttgacttgaggggtataccactggacttttcatcttcccttgggattattgtgaaagattttgagacaattggacaaaataaattaattggcacggcgactgtagccctgaaggacctgactggtgaccagagcagatccctgccgtacaagctgatctccctgctaaatgaaagagggcaagatactggggccaccattgacttggtgatcggctatgatccgccttctgctccacatccaaatgacctgagc

SEQ ID NO: 30 - DNA, 330 bp, synthetic construct
  source 1..330: mol_type = other DNA
ccacaggacttccagatccgcgtccgagtgattgagggccgacagttaagtggcaacaacataaggcctgtggtcaaagttcacgtctgtggccagacacaccgaacaagaatcaagagaggaaacaaccctttttttgatgagttgtttttctacaatgtcaacatgaccccttctgaattgatggatgagatcatcagcatccgggtttataattctcactctctgcgggcagattgtctgatgggggaatttaagattgatgttggatttgtttatgatgaacctggccatgctgtcatgagaaagtggcttcttctcaatgacccg

SEQ ID NO: 31 - DNA, 492 bp, synthetic construct
  source 1..492: mol_type = other DNA
accttcttgctgaaaatctaccgagctgaggacatcccccagatggatgatgccttctcacagacagtaaaggaaatatttggaggcaatgcagataagaaaaatctcgtggatccttttgtagaagtttcctttgctggaaaaaaggtttgtacaaacataattgagaagaatgcaaacccagagtggaatcaggtcgtcaatcttcagatcaagtttccttcagtgtgtgaaaaaataaaactaacaatatatgactgggaccgtcttactaaaaatgatgtagttggaacaacatatctacacctctctaaaattgctgcctctggtggggaagtggaagatttctcatcttcgggaactggggctgcatcatatacagtaaacacaggagaaacagaggtaggctttgttccaacgtttggaccttgttacctgaatctttatggaagccccagagagtacacgggattcccagacccctatgatgag

SEQ ID NO: 32 - DNA, 402 bp, synthetic construct
  source 1..402: mol_type = other DNA
acccccattgtttcctgcaattttgacagagtctacatctaccatctgcgctgctatgtctatcaagccagaaacctcttggctttagataaggatagcttttcagatccatatgctcatatctgtttcctccatcggagcaaaaccactgagatcatccattcaaccctgaatcccacgtgggaccaaacaattatattcgatgaagttgaaatctatggggaaccccaaacagttctacagaatccacccaaagttatcatggaactttttgacaatgaccaagtgggcaaagatgaatttttaggacgaagcattttctctcctgtggtgaaactgaactcagaaatggacatcacacccaaacttctctggcacccagtaatgaatggagacaaagcc

SEQ ID NO: 33 - DNA, 231 bp, synthetic construct
  source 1..231: mol_type = other DNA
agaaatatgaaaaacttccagatggcttctatcacatcccccagtcttgttgtggagtgtggaggagaaagggtggaatcggtggtgatcaaaaaccttaagaagacacccaactttccaagttctgttctcttcatgaaagtgttcttgcccaaggaggaattgtacatgcccccactggtgatcaaggtcatcgaccacaggcagtttgggcggaagcctgtcgtcggc

SEQ ID NO: 34 - DNA, 372 bp, synthetic construct
  source 1..372: mol_type = other DNA
cagtttcgggaattacctgacagcgtcccacaggaatgcacggttaggatttacattgttcgaggcttagagctccagccccaggacaacaatggcctgtgtgacccttacataaaaataacactgggcaaaaaagtcattgaagaccgagatcactacattcccaacactctcaacccagtctttggcaggatgtacgaactgagctgctacttacctcaagaaaaagacctgaaaatttctgtctatgattatgacacctttacccgggatgaaaaagtaggagagacaattattgatctggaaaaccgattcctttcccgctttgggtcccactgcggcataccagaggagtactgtgtttctggagtc

SEQ ID NO: 35 - DNA, 396 bp, synthetic construct
  source 1..396: mol_type = other DNA
cctttcaacatcacaccccggaaagccaagaaatactacctgcgtgtgatcatctggaacaccaaggatgttatcttggatgagaaaagcatcacaggagaggaaatgagtgacatctacgtcaaaggctgggttcctggcaatgaagaaaacaaacagaaaacagatgtccattacagatctttggatggtgaagggaattttaactggcgatttgttttcccgtttgactaccttccagccgaacaactctgtatcgttgcgaaaaaagagcatttctggagtattgaccaaacggaatttcgaatcccacccaggctgatcattcagatatgggacaatgacaagttttctctggatgactacttgggtttcctagaacttgacttgcgtcac

SEQ ID NO: 36 - DNA, 174 bp, synthetic construct
  source 1..174: mol_type = other DNA
ctgcaaacaaatatagaagctctaaaatcagggatacaaggtaaaattcctgcaaaccagctggctgaattgtggctgaagctgatagatgaagttatagaagacacgagatacacgttgcctctcacagaaggaaaagccaacgtcacagttctcgatactcagatccgaaag

SEQ ID NO: 37 - DNA, 216 bp, synthetic construct
  source 1..216: mol_type = other DNA
tggcttgataaattaatgcagctgactgaagagccacagaacagcatgcctgacatcatcatctggatgatccggggagagaagagactggcctatgcacgaattcccgcacatcaggtcttgtactccaccagtggtgagaatgcatctggaaaatactgtgggaaaacccaaaccatctttctgaagtatccacaggagaaaaacaacgggcca

SEQ ID NO: 38 - DNA, 174 bp, synthetic construct
  source 1..174: mol_type = other DNA
gctgtggagaagaagtttaacagcttcgcagaaggaactttcaccgtctttgctgaaatgtatgaaaatcaagctctcatgtttggaaaatggggtacttctggattagtaggacgtcataagttttctgatgtcacaggaaaaataaaactcaagagggaattttttctgcct

SEQ ID NO: 39 - DNA, 171 bp, synthetic construct
  source 1..171: mol_type = other DNA
gatcctgaaagaagcttgctgactgaggcagatgcaggtcacacggagttcactgatgaagtctaccagaacgagagccgctaccccgggggcgactggaagccggccgaggacacctacacggatgcgaacggcgataaagcagcatcacccagcgagttgacttgtcct

SEQ ID NO: 40 - DNA, 357 bp, synthetic construct
  source 1..357: mol_type = other DNA
ccggacctcaaagccatgaacccccttaaagccaagacagcctccctctttgagcagaagtccatgaaaggatggtggccatgctacgcagagaaagatggcgcccgcgtaatggctgggaaagtggagatgacattggaaatcctcaacgagaaggaggccgacgagaggccagccgggaaggggcgggacgaacccaacatgaaccccaagctggacttaccaaatcgaccagaaacctccttcctctggttcaccaacccatgcaagaccatgaagttcatcgtgtggcgccgctttaagtgggtcatcatcggcttgctgttcctgcttatcctgctgctcttcgtggccgtg

SEQ ID NO: 41 - protein, 386 aa, synthetic construct
MATTMGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRAASRASSRPRPDDLEI

SEQ ID NO: 42 - DNA, 3351 bp, synthetic construct
  source 1..3351: mol_type = other DNA
atggtgccagcccctcccagacagtttcgggaattacctgacagcgtcccacaggaatgcacggttaggatttacattgttcgaggcttagagctccagccccaggacaacaatggcctgtgtgacccttacataaaaataacactgggcaaaaaagtcattgaagaccgagatcactacattcccaacactctcaacccagtctttggcaggatgtacgaactgagctgctacttacctcaagaaaaagacctgaaaatttctgtctatgattatgacacctttacccgggatgaaaaagtaggagagacaattattgatctggaaaaccgattcctttcccgctttgggtcccactgcggcataccagaggagtactgtgtttctggagtcaatacctggcgagatcaactgagaccaacacagctgcttcaaaatgtcgccagattcaaaggcttcccacaacccatcctttccgaagatgggagtagaatcagatatggaggacgagactacagcttggatgaatttgaagccaacaaaatcctgcaccagcacctcggggcccctgaagagcggcttgctcttcacatcctcaggactcaggggctggtccctgagcacgtggaaacaaggactttgcacagcaccttccagcccaacatttcccagggaaaacttcagatgtgggtggatgttttccccaagagtttggggccaccaggccctcctttcaacatcacaccccggaaagccaagaaatactacctgcgtgtgatcatctggaacaccaaggatgttatcttggatgagaaaagcatcacaggagaggaaatgagtgacatctacgtcaaaggctgggttcctggcaatgaagaaaacaaacagaaaacagatgtccattacagatctttggatggtgaagggaattttaactggcgatttgttttcccgtttgactaccttccagccgaacaactctgtatcgttgcgaaaaaagagcatttctggagtattgaccaaacggaatttcgaatcccacccaggctgatcattcagatatgggacaatgacaagttttctctggatgactacttgggtttcctagaacttgacttgcgtcacacgatcattcctgcaaaatcaccagagaaatgcaggttggacatgattccggacctcaaagccatgaacccccttaaagccaagacagcctccctctttgagcagaagtccatgaaaggatggtggccatgctacgcagagaaagatggcgcccgcgtaatggctgggaaagtggagatgacattggaaatcctcaacgagaaggaggccgacgagaggccagccgggaaggggcgggacgaacccaacatgaaccccaagctggacttaccaaatcgaccagaaacctccttcctctggttcaccaacccatgcaagaccatgaagttcatcgtgtggcgccgctttaagtgggtcatcatcggcttgctgttcctgcttatcctgctgctcttcgtggccgtgctcctctactctttgccgaactatttgtcaatgaagattgtaaagccaaatgtgtacccatacgacgtcccagactacgcttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattcaaaaaaactagctgctggacatgaattacagccactagccattgtggaccagcgaccttcaagcagagccgccagtcgtgccagcagcagacctcggcctgatgacctggagatctga